(19) United States
(12) Patent Application Publication
(10) Pub. No.: US 2013/0017248 A1
(43) Pub. Date: Jan. 17, 2013

(54) Cationic Liposomes And Method of Use

(71) Applicants: Ronald Graham, Carlsbad, CA (US); Maura Barbisin, San Mateo, CA (US)

(72) Inventors: Ronald Graham, Carlsbad, CA (US); Maura Barbisin, San Mateo, CA (US)

(73) Assignee: LIFE TECHNOLOGIES CORPORATION, Carlsbad, CA (US)

(21) Appl. No.: 13/468,980

(22) Filed: May 10, 2012

Related U.S. Application Data
(63) Continuation of application No. 12/472,207, filed on May 26, 2009, now abandoned.
Application No. 12/472,207 is a continuation of application No. 11/302,584, filed on Dec. 14, 2005, now abandoned.
(60) Provisional application No. 60/636,414, filed on Dec. 14, 2004.
(60) Provisional application No. 60/636,415, filed on Dec. 14, 2004.

(51) Int. Cl. A61K 9/127

(52) U.S. Cl. 424/450

(57) ABSTRACT

Highly efficient cationic liposomes are provided as a system for the delivery to cells of agents or compounds, such as, compounds capable of silencing a target protein and enzyme stubstrates. The cationic liposomes can be used in methods of detecting the inhibition activity or apparent activity of a target protein in a cell, and methods of identifying a protein associated with a pathway, such as, a signal transduction pathway in a cell.

### 1. CROSS REFERENCE TO RELATED APPLICATIONS

This application is a continuation of U.S. application No. 12/472,207, filed May 26, 2009, which is a continuation of U.S. application No. 11/302,584, filed Dec. 14, 2005, and claims priority of U.S. application No. 60/636,414, filed Dec. 14, 2004 and U.S. application No. 60/636,415, filed Dec. 14, 2004, which disclosures are herein incorporated by reference in their entirety.

### 2. BACKGROUND

Currently, methods of delivering agents to cells include hypotonic shock, electroporation, calcium-phosphate-based transfection, infectious agents and liposomes. Liposomes are vesicles of one or more phospholipid bilayers separated by equal numbers of aqueous interspaces, which may contain or complex virtually any type of agent. Accordingly, liposomes are useful as in vitro and in vivo delivery systems for, inter alia, therapeutic agents, diagnostic agents, and analytical agents. Although numerous liposome compositions are known in the art, significant problems such as toxicity and inefficient agent delivery still exist.

Accordingly, there is a need for new liposome compositions of reduced toxicity that can efficiently deliver a wide variety of agents to cells, including agents for rapid assessment of target proteins in living cells.

### 3. SUMMARY

These and other features of the present invention are set forth below.

In some embodiments, a cationic liposome is provided. The liposome can comprise a charge neutral compound and/or a charge neutral mixture of compounds and a cationic phospholipid in which the molar ratio of the charge neutral compound and/or charge neutral mixture of compounds to cationic phospholipid is greater than about 1:1.

In some embodiments, a method of delivering an agent to a cell is provided. The cell can be contacted with a cationic liposome which complexes or encapsulates one or more agents. A cationic liposome can comprise a charge neutral compound and/or a charge neutral mixture of compounds and a cationic phospholipid in which the molar ratio of the charge neutral compound and/or charge neutral mixture of compounds to cationic phospholipid is greater than about 1:1 along with the agent.

In some embodiments, a liposome can comprise (i) a compound capable of silencing a target protein and (ii) an enzyme substrate, wherein the liposome is capable of delivering the compound and the enzyme substrate into a live cell. In some embodiments, the compound can be capable of controlling the apparent activity of a target protein. In some embodiments, the enzyme substrate can be capable of producing a detectable signal when modified by a readout protein.

In some embodiments, a method disclosed herein comprises detecting the inhibition of expression of a target protein in a live cell. In some embodiments, a method comprises contacting a cell with a liposome comprising (i) a compound capable of silencing a target protein and (ii) an enzyme substrate capable of producing a detectable signal when modified by a readout protein. In some embodiments, a method comprises detecting a change in signal which indicates the inhibition of expression of said target protein in the cell, in some embodiments, a method disclosed herein comprises identifying a target protein as being associated with a pathway, such as an enzymatic or signal transduction pathway, in a living cell. In some embodiments, a method comprises contacting a cell with a liposome comprising (i) a compound capable of silencing a target protein (ii) an enzyme substrate capable of producing a detectable signal when modified by a readout protein; contacting the cell with an agonist of the pathway and detecting whether a detectable signal is produced. The change in detectable signal indicates that the target protein is associated with the signal transduction pathway.

Further provided are kits for use in delivering an agent to a cell. In some embodiments, a kit can contain a charge neutral compound and/or a charge neutral mixture of compounds and a cationic phospholipid and instructions to generate- a cationic liposome which delivers the agent to the cell.

Further provided are kits for use in detecting the apparent activity of a target protein. In some embodiments, a kit can be used for detecting the :inhibition of expression of a target protein in a cell, comprising lipids capable of forming a cationic liposome, compounds capable of silencing a target protein and an enzyme substrate capable of producing a detectable signal when modified by an enzyme.

### 4. BRIEF DESCRIPTION OF THE FIGURES

The drawings described below are for illustration purposes only and are riot intended to limit the scope of the present teaching in any way.

FIG. 1 shows exemplary embodiments of a liposome comprising a target protein silencing compound (“C”) and an enzyme substrate (“S”) (Panel A) and the liposome releasing C and S into a cell (Panel B),

FIG. 2 shows exemplary embodiments in which the apparent activity of target protein (“TP”) can be controlled.

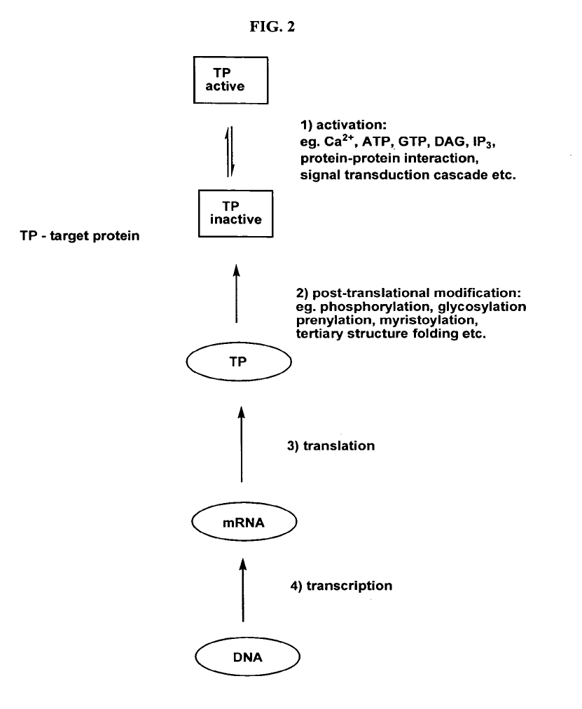

FIG. 3 shows a schematic signal transduction cascade showing direct and indirect measurement of the apparent activity of a target protein.

FIG. 4, Panels A, B, C, and D show an exemplary embodiments of cationic liposome delivery of labeled phalloidin to HeLa cells.

FIG. 5, Panel A shows an exemplary embodiment of cationic liposome delivery of labeled phalloidin to HeLa cells with Hoechst nuclear staining. Panel B is a reproduction of Panel A in which areas of blue fluorescence (B) are indicated. Other areas of fluorescence are green.

### 5. DETAILED DESCRIPTION

It is to be understood that both the foregoing summary and the following description of various embodiments are exemplary and explanatory only and are not restrictive of the present teachings. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting. The term “delivery” in the context of the present disclosure denotes the introduction of agents, into a cell, in vivo or in vitro.

5.1 Definitions

As used herein, the following terms are intended to have the following meanings:

“Alkanyl” by itself or as part of another substituent means a saturated branched, straight-chain or cyclic alkyl derived by the removal of one hydrogen atom from a single carbon atom of a parent alkane. Typical alkanyl groups include, but are not limited to, methanyl; ethanyl; propanyls, such as, propan-1-yl, propan-2-yl(isopropyl), cyclopropan-1-yl, etc.; butanyls, such as, butan-1-yl, butan-2-yl(sec-butyl), 2-methyl-propan-1-yl(isobutyl), 2-methyl-propan-2-yl(t-butyl), cyclobutan-1-yl, etc.; and the like.

“Alkenyl” by itself or as part of another substituent means an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls, such as, prop-1-en-1-yl, prop-1-en-2-yl, prop-2-en-1-yl, prop-2-en-2-yl, cycloprop-1-en-1-yl; cycloprop-2-en-1-yl; butenyls, such as, but-1-en-1-yl, but-1-en-2-yl, 2-methyl-prop-1-en-1-yl, but-2-en-1-yl, but-2-en-2-yl, but-1,3-dien-1-yl, buta-1,3-dien-2-yl, cyclobut-1-en-1-yl, cyclobut-1-en-3-yl. cyclobut-1,3-dien-1-yl, etc.; and the like.

“Alkynyl” by itself or as part of another substituent means an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls, such as, prop-1-yn-1-yl, prop-2-yn-1-yl, etc.; butynyls, such as, but-1-yn-1-yl, but-1-yn-3-yl, but-3-yn-1-yl, etc.; and the like.

“Acyl” refers to a radical —C(O)R, where R is hydrogen or alkyl as defined herein. Representative examples include, but are not limited to formyl, acetyl and the like.

“Salt” refers to a salt of a compound described herein which possesses the desired activity of the parent compound. Such salts include: (1) acid addition salts, formed with inorganic acids, such as, hydrochloric acid, hydrobromic acid, sulfuric acid, nitric acid, phosphoric acid, and the like; or formed with organic acids, such as, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.21-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base, such as, ethanolamine, diethanolamine, triethanolamine, N-methylglucamine and the like.

5.2 Cationic Liposomes

The present disclosure provides cationic liposomes that can deliver a wide variety of agents (e.g., therapeutic agents, diagnostic agents, etc.) to cells. Cationic liposomes include a charge neutral compound and/or a charge neutral mixture of compounds and a cationic phospholipid. In some embodiments the, liposomes can be of reduced toxicity compared with other types of liposomes.

In some embodiments, the molar ratio of charge neutral compound and/or charge neutral mixture of compounds can be greater than the cationic phospholipid. In some embodiments, the molar ratio of charge neutral compound and/or charge neutral mixture of compounds to cationic phospholipid can be greater than about 1:1 to about 10:1. Thus, in various exemplary embodiments, the molar ratio of charge neutral compound and/or charge neutral mixture of compounds to cationic phospholipid can be about 2:1, about 3:1, about 4:1, about 5:1, about 6:1, about 7:1, about 8:1, about 9:1, or less than about 10:1.

A charge neutral mixture of compounds can be any mixture of anionic, cationic or neutral compounds with a net charge that is about zero. The ratio of the individual compounds is unimportant as long as the mixture, as a whole, has a net charge that is about zero.

A wide variety of charge neutral compounds and/or charge neutral mixture of compounds may be used to form the cationic liposomes described herein. Charge neutral compounds and/or charge neutral mixture of compounds include, but are not limited to, lipids, phospholipids, pegylated phospholipids, cholesterols, steroids, tocopherols, nitroxides and combinations thereof.

Generally, a phospholipid can be amphiphilic. In some embodiments, amphilic phospholipdis have two hydrophobic fatty acid tails and a hydrophilic head. The hydrophilic head can include a glycerol backbone, a phosphate and a polar moiety. Some phospholipids, for example, phosphotidylcholine have both a cationic polar moiety (+1) and a negatively charged phosphate (−1) with a total net charge of zero. Masking of the negative charge of the phosphate group provides a cationic phospholipid.

In some embodiments, a phospholipid can be a (mono/di)radylglycerophospho-(monohydroxyalcholol) such as, for example, a diacylglycerophospholipid, an alk(en)ylacylglycerophospholipid, a dialk(en)ylglycerophospholidpid, a monoacyl-glycerophospholipid and a monoalkylglycerophsopholipid. Diacylglycero-phospholipids are phosphodiester derivatives of 1,2-diacyl-sn-glycero-3-phosphate, such as, for example, 1,2-dihexadecanoyl-sn-glycero-3-phosphocholine. Alk(en)ylacyl glycerophospholipids or dialk(en)yl glycerophospholipids include one or two alkyl or alkenyl chains, such as, for example, 1-hexadecyl-2-acetyl-sn-glycero-3-phosphocholine and 1-(1′-glyceroalkyl)-2-acyl-sn-glycero-phosphoethanolamines. Monoacyl-glycerophospholipids or monoalkyl-glycerophospholipid, include, for example, 2-hexadecanoyl-sn-glycero-3-phosphocholine and 1-hexadecyl-sn-glycero-3-phosphocholine.

In some embodiments, a phospholipid can be a (mono/di)radylglycerophospho-polyol. Suitable examples include, but are not limited to, 1,2-diacyl-sn-glycero-3-phospho derivatives of glycerol and D-myo-inositol, such as, phosphatidylglycerol and phosphatidylinositol, respectively. Phospholipids of this type include, but are not limited to, multiple polyol moieties such as, for example, diacylphosphatidylglycerol (1-(1′2′-diacyl-sn-glycero-3′-phospho)-sn-glycerol), cardiolipon(1,3-bis(1′2′-diacyl-sn-glycero-3′-phospho)-glycerol, lyso-bisphosphantidic acid, (1-(3′-acyl-sn-glycerol-1′-phospho)-3-acyl-sn-glycerol, phosphatidylinositol and (1-(1′2′-diacyl-sn-glycero-3′-phospho)-L-myo-itiositol.

In some embodiments, a phospholipid can be a (mono/di)radylglycerolglycoside. The phospholipid may be, for example, a glycoglycerolipid bearing a phospho-, a glycerophospho-, or a mono- or diradylglycerophospho residue. Exemplary phosphoglycolipids include, but are not limited to, glycerophosphomonoglucosyl phospholipids, glycerophosphodiglucosyl phospholipids, 3′-O-glucosaminyl-phosphatidylglycerol and dimannosol-inositol phospholipids.

In some embodiments, a phospholipid can be a (mono/di)radylglycero-phosphoglycoside. Such phospholipids can be glycosylated derivatives of phosphatidylglycerol and phosphatidylinositol described, herein. The phospholipid may be glycosylated with well known glycosyl groups such as, for example, glucose, mannose, galactose, aminoglucose, N-acetyl aminoglucose; and N-acetyl aminogalactose etc.

In some embodiments, a phospholipid can be a sphingosine-containing phospholipid. Sphingosine-containing phospholipids include, for example, sphingomyelins (i.e., phosphocholine derivatives of ceramides) and phytoglycolipids (i.e., glycosylated derivatives of inositol phosphoceramides).

In various exemplary embodiments, a phospholipid can be a phosphono derivative of a (mono/di)radylglycerophospho-monohydroxy alcohol, a (mono/di)radyl-glyeerophospho-polyol, a (mono/di)radylglyceroglycoside, a (mono/di)radyl-glycerophosphoglycoside or combinations thereof, such as, those described herein.

In various exemplary embodiments, a phospholipid can be acylphophatidylethanolamine, lysophophatidylethanolamine, acylphophatidyleholine, lysophophatidylcholine, acylphophatidyiserine, lysophophatidylserine, acylphophatidylglycerol, lysophophatidylglycerol, acylphophatidic acid, lysophophatidic acid, acylphophatidylinositol, lysophophatidylinositol, acylphophatidylinositol-4 -phosphate, lysophophatidylinositol-4-phosphate, lysophophatidylinositol-4,5-diphosphate, acylphophatidylinositol-4,5 -diphosphate, sphingomyelin or combinations thereof.

In some embodiments, a phospholipid is a compound of structural Formula (I):

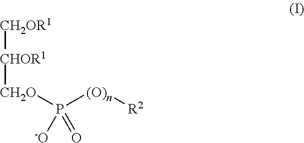

or salts, solvates or hydrates thereof wherein;

each R¹ is independently hydrogen, alkyl or acyl;

n is 1 or 0;

R² is hydrogen, —CH₂CH₂N(CH))₃, —CH₂CH₂NI4₃, —CH₂CH(CO₂-)NH₃⁺,

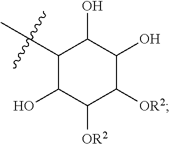

—CH₂CH(OH)CH₂OH or and

each le is independently hydrogen or —PO_{3A}H with the proviso that at least one R¹ is not hydrogen and is (C₁₀-C₃₀)alkyl or (C₁₀-C₃₀)acyl.

In some embodiments, n is 1 and R² is hydrogen, —CH₂CH₂N(CH₃)₃, —CH₂CH₂NH₃, —CH₂CH(CO₂—)NH₃⁺ or —CH₂CH(OH)CH₂OH. In some embodiments n is 1 and R¹ is alkyl or acyl. In some embodiments, n is 1 and one R¹ is hydrogen. In some embodiments, n is 1 and the R¹ group attached to the secondary hydroxyl is hydrogen. In some embodiments, n is 1 and R¹ is hydrogen or acyl. In some embodiments each R¹ is identical. In some embodiments, each R¹ is independently capryl, undecanoyl, lauroyl, tridecanoyl, myristoyl, pentadecanoyi, palmitoyl, phytanoyl, heptadecanoyl, stearoyl, nonadecanoyl, arachidoyl, heneicosanoyl, trieosanoyl, lignoceroyl, myristoleoyl, myristelaidoyl, palmitoleoyl, palmitelaidoyl, petroselinoyl, oleoyl, elaidoyl, linoleoyl, linolenoyi, eicosenoyl, arachidonoyl, erucoyl or nervonoyl. In some embodiments, a phospholipid can be 1,2-dioleoyl-sn-glycero-3-phosphocholine.

In some embodiments, a cationic phospholipid can include a protecting group attached to the negatively charged oxygen of the phosphate group. A protecting group, as is known to those of skill in the art, refers to a grouping of atoms that when attached to a functional group in a molecule (e.g, the negatively charged oxygen of the phosphate oxygen) masks the reactivity of the functional group. Examples of protecting groups can he found, for example, in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Exemplary, phosphate protecting groups include, but are not limited to, those where the phosphate oxygen is either acylated or allcylated, such as, acetyl, benzyl, trityl ethers, alkyl ethers, tetrahydropyranyl ethers, trialkylsilyl ethers, allyl ethers, etc. In some embodiments, the phosphate protecting group can be ethyl, acetoxymethyl or S-acyl-2-thioethyl.

In some embodiments, a protected cationic phospholipid can be biolabile under conditions (e.g, bioassay conditions, physiological conditions, etc.) including conditions described herein. Generally, a biolabile protected phospholipid can be deprotected (i.e., loses its phosphate protecting group) to provide a charged phosphate group.

In various exemplary embodiments a cationic phospholipid can be a protected (mono/di)radylglycerophospho-monohydroxy alcohol, a protected (mono/di)radylglyceroglycoside, a protected (mono/di)radylglycerophosphoglycoside, sphingosine, a protected phosphono derivative of a (mono/di)radylglycerophospho-monohydroxy alcohol, a protected (mono/di)radylglyceroglycoside, a protected (mono/di)radylglycerophosphoglycoside or combinations thereof.

In various exemplary embodiments, a cationic phospholipid can be .a protected acylphophatidylethanolamine, lysophophatidylethariolamine, acylphophatidylcholine, lysophophatidylcholine, acylphophatidylserine, lysophophatidylserine, acylphophatidylglycerol, lysophophatidylglycerol, acyiphophatidic acid, lysophophatidic acid, acylphophatidylinositol lysophophatidylinositol, acylphophatidylinositol-4-phosphate, lysophophatidylinositol-4-phosphate, lysophophatidylinositol-4,5-diphosphate, acylphophatidylinositol-4,5-diphosphate or sphingomyelin.

In some embodiments, a cationic phospholipid is a compound of structural formula (II):

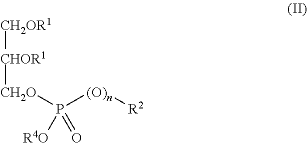

or salts, solvates or hydrates thereof wherein;

each R¹ is independently hydrogen, alkyl or acyl;

n is 0 or 1;

R² is —CH₂CH₂N(CH₃)₃, —CH₂CH₂NH₃, and

R⁴ is a protecting group;

with the proviso that at least one R¹ is not hydrogen and is (C₁₀-C₃₀)alkyl or (C₁₀-C₃₀)acyl.

In some embodiments, R⁴ is —R⁵, —CH₂OC(O)R⁵ or —CH₂CH₂ SC(O)R⁵ wherein R⁵ is (C₁-C₆)alkyl. In some embodiments, n is 1 and R² is —CH₂CH₂N(CH₃)₃. In some embodiments, n is 1 and R² is —CH₂CH₂N(CH₃)₃. In some embodiments, n is 1 and R¹ is hydrogen or acyl. In some embodiments, n is 1 and R¹ is alkyl or acyl. In some embodiments, n is 1 and R¹ is hydrogen. In some embodiments, n is 1 and the R¹ group attached to the secondary hydroxyl is hydrogen, in some embodiments, n is 1, le is alkyl or acyl and R² is —CH₂CH₂N(CH₃)₃. In some embodiments, a cationic phospholipid can be 1,2-dioleoyl-sn-glycero-3-ethylphosphocholine. In some embodiments, both R¹ groups are identical. In some embodiments, each R¹ is independently capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, tricosanoyl, Iignoceroyl, myristoieoyl, myristelaidoyl, palmitoleoyl, palmitelaidoyl, petroselinoyl, oleoyl; elaidoyl, linoleoyl, Iinoienoyl, eicosenoyi, arachidonoyl, erucoyl and nervonoyl.

In some embodiments the cationic liposome comprises 1,2-dioleoyl-sn-glycero-3-phosphocholine and 1,2-dioleoyl-sn-glycero-3-ethylphosphocholine. In some embodiments, the molar ratio of 1,2-dioleoyl-sn-glycero-3-phosphocholine is greater than the molar ratio of 1,2-dioleoyl-sn-glycero-3-ethylphosphocholine. In some embodiments, the molar ratio of 1,2-diolcoyl-sn-glycero-3-phosphocholine to 1,2-dioleoyl-sn-glycero-3-ethylphosphocholine is in the range of greater than about 1:1 to about 10:1 In various exemplary embodiments, the molar ratio of 1,2-dioleoyl-sn-glycero-3-phosphocholine to 1,2-diolcoyl-sn-glycero-3-ethylphosphocholine can be about 2:1, about 3:1, about 4:1, about 5:1, about 6:1, about: 7:1, about 8:1, about 9:1, to less than about 10:1.

In some embodiments a liposome can be biodegradable.

In sonic embodiments, a liposome can be cationic and include a 1,2-diacyl-sn-glycero-3-allcylphosphocholine having formula (III):

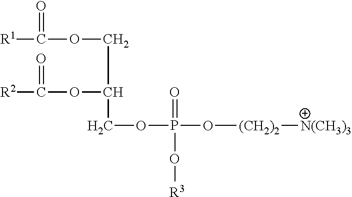

wherein:

R¹ is a saturated or unsaturated alkyl having from 6 to 30 carbon atoms;

R² is a saturated or unsaturated alkyl having from 6 to 30 carbon atoms;

R³ is a saturated or unsaturated alkyl having from 1 to 20 carbon atoms.

In some embodiments, a liposome can include 1,2-dioleoyl-sn-glycero-3-ethylphosphocholine. In some embodiments, the liposome can include both 12-dioleoyl-sn-glycero-3-ethylphosphoeholine and 1,2-dioleoyl-sn-glycero-3-phosphocholine. In some embodiments, a liposome can include these two phospholipids in a molar ratio in the range of about 1;10 to about 10:1. in some embodiments, the molar ratio is about 1:2. The liposome may also include other lipids or components as described herein.

The compounds disclosed herein may contain one or more chiral centers and/or double bonds and therefore, may exist as stereoisomers, such as, double-bond isomers (i.e., geometric isomers), enantiomers or diastereomers. Accordingly, the chemical structures depicted herein encompass all possible enantiomers and stereoisomers of the disclosed compounds including the stereoisomerically pure form (e.g., geometrically pure, enantio:merically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

The compounds disclosed herein may also exist in various tautomeric forms including the enol form, the keto form and mixtures thereof. Accordingly, the compounds herein encompass all possible tautomeric forms.

The compounds disclosed herein may exist in various unsolvated and solvated forms, such as, hydrated forms, and as N-oxides. The compounds disclosed herein may exist in multiple crystalline or amorphous forms, in general, all physical forms are equivalent tbr the uses contemplated herein.

The compounds disclosed herein may exist as various isotopic forms. Examples of isotopes that may be incorporated into the compounds disclosed herein include, but are not limited to, ²H, ³H, ¹³C, ¹⁴C, ¹⁵N, ₁₈O, ¹⁷O, ³¹P, ¹⁸F and ³⁶Cl.

In various exemplary embodiments, cationic Liposomes can include cholesterol, cholesterol derivatives, steroids and tocols. Steroids include bile acid and sterol derivatives such as, for example, cholate, ursodeoxycholate, chenodeoxycholate, taurochenodeoxycholate, tatiroursocleoxycholate, glycochenodeoxycholate, glycoursodeoxycholate, sterols and sterol esters or ethers, such as, PEG-24 cholesterol ether (Solulart® C-24). Tocol derivatives include derivatives of substances with the tocol structure[2-methyl-2-(4,8,12-trimethyltridecycl)chroman-6-ol] or the tocotrienol structure [2-methyl-2-(4,8,12-trimethyltrideca-3,7,11-trienyl)chroman-6-ol]. In particular, the mono-, di-, triniethyl-tocols, commonly known as tocopherols and their organic acid esters, such as, the acetate, nicotinate, succinate, and polyethylene glycol succinate esters are included. For example, α-tocopherol acetate, α-tocopherol nicotinate, α-tocopherol succinate, u-tocopherol polyethylene glycol (200-8000 MW) succinate, α-tocopherol polyethylene glycol 400 succinate, α-tocopherol polyethyleneglycol 1000 succinate (Vitamin E-TPGS, Eastman Chemical Co.) are included as mixed racemic di-forms, and the pure d- and l-enantiomers. The concentration of cholesterol, cholesterol derivatives, steroids and tocol derivatives in liposomes can be in the range, for example, of between about 5 mol % to about 60 mol %, although higher or lower concentrations can be used.

In various exemplary embodiments, cationic liposomes can include saturated and unsaturated lipids such as, for example, sphingosine, ceramide, cerebroside, detergents, surfactants, soaps and combinations thereof. Lipids include synthetic lipid compounds, such as, D-erythro (C-18) derivatives including sphingosine, ceramide derivatives, and sphinganine; glycosylated (C-18) sphingosine and L-threo (C-18) derivatives, all of which are commercially available (Avanti Polar Lipids, Alabaster, Ala.). Detergents include, but are not limited to, α-tocopherol polyethylene glycol succinate (TPGS), PS-80, sodium cholate, sodium dodecylsulfate, sodium salts of N-lauroylsarcosine, lauryldimethylamine oxide, cetylfrimethylammonium bromide and sodium salt of bis(2-ethylhexyl)sulfosuccinate.

A wide variety of suitable lipids are commercially available (such as from Avanti Polar Lipids, Inc., Alabaster, Ala.; Boehringer-Mannheim; Promega; Life Technologies (Gibco)). Non-limiting examples of suitable lipids include 1,2-dimyristoyl-sn-glyeero-3-phosphate (Monosodium Salt), (1,2-dipalmatoyl-sn-glyeero-3-phosphate (Monosodium Salt), and 1,2-dioleoyl-3-trimethylammonium propane (Chloride Salt). Commercially available lipids can be obtained in kits, such as, LIPOFECTIN™, LIPOFECTAMINE™, LIPOFECTACE™, ELLFECTIN™, TRANSFECTAM™, TRX-50™, DC-CHOL™ and DOSPER™ (Lasic, Liposomes in Gene Delivery, CRC Press, New York p. 86).

In some embodiments, a cationic liposome can be used as a targeting system to deliver specific exogenous agents to specific cells or specific portions of cells, such as, the cytoplasm, the nucleus, and/or other organelles. For example, organic compounds of less than about 1000 MW and/or proteins or peptides which bind to a cell surface or subcellular compartment may be included in the liposomes described herein to localize delivery. In some embodiments, a cationic liposome may include a ligand or ligand like component for a specific cell surface receptor or nuclear receptor. In some embodiments, a ligand such an antibody, hormone, carbohydrate, growth factor, a neurotransmitter, or fragments thereof or a nuclear localization signal may be included in a cationic liposome to localize delivery. Further selectivity can be achieved by incorporating into the liposome specific molecules, such as, antibodies, lectins, peptides/proteins, carbohydrates, glycoproteins, and the like, which serve to “target” the liposome to the desired receptor or binding site of the specific molecule.

In some embodiments, fusion proteins can be incorporated into a cationic liposome to form a fusigenic liposome. Fusigenic liposomes efficiently fuse with cellular membranes and can be prepared by coupling various proteins with the liposomes. For example, fusigenic liposomes can comprise one or more proteins from a virus, such as, a paramyxovirus (e.g, respiroviruses (e.g, Sendai virus, hemagglutinating virus of Japan (HVJ)) (Dzau et al. 1996 Proc. Natl. Acad. Sci. USA 93:11421-11425).

Cationic liposomes disclosed herein and other types of liposomes can be prepared using various methods and can have various sizes and can have one or more lamallae (e.g, Lasic, Liposomes in Gene Delivery, CRC Press, New York pp. 67-112 (1997), Ann. Rev. Biophys. Bioeng. 9:467-508 (1980); European Patent Application 0172007; U.S. Pat. Nos. 4,229,360; 4,241,046; 4,235,871; 5,455,157; 6,284,538; 6,458,381; and 6,534,018). Many preparation methods involve steps, such as, preparation of the lipid for hydration, followed by hydration with agitation (e.g, extrusion, sonication, and/or homogenization), and sizing to a homogeneous distribution of liposomes, for example, although any suitable preparation method may be used. Properties of liposomes can vary depending on their composition (cationic, anionic, neutral lipid species), however, the same preparation method can be used for all liposomes regardless of composition. In some embodiments, liposomes of various sizes and shapes can be prepared, such as, large multilamellar vesicles (LMV), unilamellar vesicles, small (SUV), large (LUV), or giant (GUV) vesicles. In some embodiments, a suitable preparation can have a heterogeneous size distribution. Alternatively, a suitable preparation can comprise a substantially uniform or narrow size distribution. For example, liposomes having a diameter in the range of about 50 nm to about 250 nm can be prepared, although other sizes are possible. In some embodiments in which substrates or other agents are encapsulated into liposomes, conventional methods can be used for loading, such as, reverse phase methods and sonication (e. g, as described by Lasic (1997) p. 93 and in U.S. Pat. No. 4,888,288). After loading, the liposomes can optionally be subjected to dialysis or molecular sieving (e,g, by Q Sepharose separation). In some embodiments, substrates may be encapsulated during liposome preparation, such as, described in herein. In some embodiments, a substrate is provided in a form that is at least about 80%, 85%, 90%, 95%, 96%, 97%, 98%, or 99% liposomal as opposed to free in solution. To enhance stability, a liposome preparation may be stored in the dark, under argon, and at a low temperature, such as, 4° C., for example.

In selecting components for preparing cationic liposomes disclosed herein and other types of liposomes for use with various cells types, one or more tests can be performed to confirm that cells are viable after contact with the liposomes. Any conventional test for viability can be used. For example, dye exclusion methods can be used. Trypan Blue is a blue stain which normally does not substantially penetrate the plasma membrane and therefore is substantially excluded from viable cells. Only cells with damaged plasma membrane take on a blue color. The stained and unstained cells can be counted in a heinacytometer with a standard light microscope and the percent viability can be calculated. In another example, propidium iodide (PI) can be used in a similar manner. PI can substantially penetrate damaged membranes, interacts with DNA-RNA to produce an adduct that produces red fluorescence. Fluorescein diacetate (FDA) is a non-polar, non-fluorescent fluorescein analogue which upon entering a cell serves as a subsrate for intracellular esterases which remove diacetate group thereby yielding fluorescein. Fluorescein accumulates in cells which possess intact membranes and therefore green fluorescence is a marker of cell viability or metabolically active cells. (Breeuwer et ol., 1995, Appl. Environ. Microbial. 61:1614-1619; Widhohn, 1972, Stain Technol. 47:189-194). Cells can be tested for viability at any time point, such as, before, during or after an enzyme assay, and any change in viability can be determined. In some embodiments, the viability of the cells after contact with a liposomal composition can decrease by less than about 20%, less than about 15%, less than 10%, less than 5%, less than about 4%, less than about 3%, less than about 2%, or less than about 1%.

In some embodiments standard proliferation testa may be used to investigate the cytotoxicity of a cationic liposome as disclosed herein and other types of kiposomes. An exemplary test is the tetrazolium salt based calorimetric test that detects viable cells exclusively. Living, metabolically active cells substantially reduce tetrazolium salts to colored formazan compounds, whereas dead cells do not. This test can be performed in a microtitre plate after the treatment of cells with a selected liposome formulation. The colorimetric change in a sample versus control can be easily measured with a spectrophotometer. A cytotoxic factor will reduce the rate of tetrazolium salt cleavage by a population of cells.

In some embodiments, cell viability can be checked by observation of cell morphology (e.g., with a standard light in icro.scope). For example, healthy HeLa cells appear polygonal and are adherent to the surface of the vessel in which they are contained, whereas damaged cells tend to shrink, roundup, detach and float in the medium. The number of cells that appear polygonal remain attached under a selected set of conditions can be used as another measure of toxicity. Cells can be further analyzed, such by use of one or more staining methods as described herein.

The cationic liposomes described herein can encapsulate and/or complex with a wide variety of compounds or agents which can be delivered to cells. In some embodiments, a cationic liposome can encapsulate and/or complex with one agent. In some embodiments, a cationic liposome can encapsulate andlor complex with more than one agent. Non-limiting examples of agents include therapeutic agents, diagnostic agents, agents capable o r silencing a target protein, and an enzyme substrate capable of producing a detectable signal.

Therapeutic agents which may be delivered with cationic liposomes, include, for example, natural and synthetic agents with the following therapeutic activities: anti-arthritic, anti-arrhythmic, antibacterial, anticholinergic, anticoagulant, antidiuretic., antidote, anti-epileptic, antifungal, anti-inflammatory, antimetabolic, antimigraine, antineoplastic, antiparasitic, antipyretic, antiseizure, antisera, antispasmodic, analgesic, anesthetic, β-blocking, biological response modifying, bone metabolism regulating, cardiovascular, diuretic, enzymatic, fertility. enhancing, growth promoting, hemostatic, hormonal, hormonal suppressing, hypercalcernic alleviating, hypocalcemic alleviating, hypoglycemic alleviating, hyperglycemic- alleviating. immunosuppressive, immuno-enhancing, muscle relaxing, neurotransmitting, parasympathomimetic, sympathominetric plasma extending, plasma expanding, psychotropic, thrombolytic and vasodilating,

In various exemplary embodiments, therapeutic agents include cytotoxic agents, anthracycline antibiotics, such as, doxorubicin, daunorubicin, epirubicin and idarubicin, and analogs of these, such as, epirubidin and mitoxantrone; platinum compounds, such as, cisplatin, carboplatin, ormaplatin, oxaliplatin, zeniplatin, enloplatin, lobaplatin, spiroplatin, ((−)-(R)-2-aminomethylpyrrolidine (1,1-cyclobutane dicarboxylato)-platinum) (SP-4-3(R)-1,1-cyclobutane-dicarboxylato(2-)-(2-methyl-1,4-butanediamine-N,N)platinum)nedaplatin(bis-acetato-ammine-dichlorocyclohexylamine-planum(IV), vinea alkaloids, such as, vincristine, vinblastine, vinleurosine, vinrodisine, vinorelbine (navelbine) and vindesine and camptothecin and its analogues, including SN-38 ((+)-(4S)-4,11-diethyl-4,9-dihydroxy-1H-pyrano]3′,4′:6,71-indolizino[1,2-b]quindine-3,14(4H,12H)-dione); 9-aminocamptothecin; 9-nitrocamptothecin, topotecan (hycamtin 9-dimethyl-aminomethyl-10-hydroxycamptothecin); irinotecan (CPT-11; 7-ethyl-10-[4-(1-piperidino)-1-piperidino]-carbonyloxycamptothecin), 7-ethylcamptothecin and its 7-chloromethyl-10,11-methylene-dioxy-camptothecin; and others.

In various exemplary embodiments, therapeutic agents include angiotensin-converting enzyme inhibitors, such as, alecapril, captopril, 1-[4-carboxy-2-methyl-2R,4R-pentanoyl]-2,3-dihydro-2S-indole-2-carboxylic acid, enalaprilic acid, lisinopril, N-cyclopentyl-N-[3-[(2,2-dimethy)-1-oxopropyl)thio]-2-methyl-1-oxopropyl]glycine, pivopril, quinaprilat, (2R, 4R)-2-hydroxyphenyl)-3-(3-mercaptopropionyl)-4-thiazolidincarboxylic acid, (S) benzamido-4-oxo-6-phenylhexenoyl-2-carboxypyrrolidine, and tiopronin; eephalosporin antibiotics, such as, celaclor, celadroxil, celantandole, celatrizinc, cafazedone, cefazuflur, cefazolin, ccfbuperazone, cefixime, cefmenoxime, cefinetazole, cefodizime, cefonicid, cefoperazone, ceforanide, cefotaxime, cefotefan., cefotiam, cefoxitin, cefpitnizole, cefpirome, cefpodoxime, cefroxadine, cefsulodin, cefpiramide, ceftazidime, ceftezole, ceftizoxime, ceftriaxone, cefuroxime, cephacetrile, cephalexin, cephaloglycin, cephaloridine, cephalosporin, cephanone, cephradine and latamoxef; penicillins, such as, airtoxycillin, ampicillin, apalcillin, azidocillin, azlocill in, benzylpencillin, carbenicillin, carindacillin, cyclacillin, dicloxacillin, epicillin, flucloxacillin, hetacillin, methicillin, mezlocillin, nafalin, oxacillin, phenethicillin, temocillin and ticarcillin; thrombin inhibitors, such as, argatroban, melagatran and napsagatnui; influenza neuraminidase inhibitors, such as, zanamivir and BCX-1812; non-steroidal anti-inflammatory agents, such as, acametacin, alclofanac, alminoprofen, aspirin (acetylsalicylic acid), 4-biphenylacetic acid, bucloxic acid, carprofen, cinchofen, cinnietacin, clometacin, clonixin, dicIenofac, diflunisal, etodolac, fenbufen, fenclofenac, fencIosie acid, fertoprofen, ferobufen, flufenamic acid, flufenisal, flurbiprofin, fluprofen, flutiazin, ibufenac, ibuprofen, indomethaein, indoprofen, ketoprofen, ketorolac, ionazolac, loxoprofert, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprolenic acid, tolfenamic acid, tolmetin and zopemirac; prostaglandins, such as, ciprostene, 16-deoxy-16-hydroxy-16-vinyl prostaglandin E₂, 6,16-dimethylprostaglandin E₂, epoprostostenol, metencprost, nileprost, prostacyclin, prostaglandins E₁, E₂, or F_{2α} and thronthoxane A₂; and quinolone antibiotics, such as, acrosoxacin, cinoxacin, ciprofloxacin, enoxacin, flumequine, naladixic acid, norfloxacin, ofloxacin, oxolinic acid, pefloxacin, pipernidic acid and piromidic acid; other antibiotics, such as, aztreonam, imipenern, meropenern and related carbopenern antibiotics, acebutalol, albuterol, alprenolol, atenolol, bunolol, bupropion, butopamine, butoxamine, earbuterol, cartelolol, colterol, deterenol, dexpropanolol, diacetolol, dobutarnine, exaprolol, exprenolol, fenoterol, fenyripol, labotolol, levobunolol, metolol, metaproterenol, inetoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfinicdol, sulfinteroI, sulictidil, tazaolol, terbutaline, timolol, ttprenolol, tipridil, tolarnolol, thiabendazole, albendazole, albutotn, alendronate, alinidine, al izapride, am amide, aminorex, aprinocid, cambendazole, cimetidine, cisapride, clonidine, cyclobenzadole, delavirdine, efegatrin, etirttidine, fenbendazole, fenmetazole, flubendazole, findorex, gabapentin, icadronate, Iobendazole, mebendazole, metazoline, metoclopramide, methylphenidate, rnexiletine, neridronate, nocodazole, oxfendazole, oxibendazole, oxmetidine, parnidronate, parbendazole, pramipexole, prazosin, pregabalin, procainamide, ranitidinc, tetrahvdrazoline, tiamenidine, tinazoline, tiotidine, tocainide, tolazoline, trarnazol ine, xylometazoline, dimetboxyphenethylamine, N-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, carbidopa, clorprenaline, chlortermine, dopamine, L-Dopa, ephrinephrine etryptatnine, fenflurantine, methyldopamine, norepinephrine, tocainide, enviroxime, nifedipine, nimodipine, triatnterene, pipedemic acid and similar compounds, 1-ethyl-6-fluoro-1,4-dihydro-4-oxo-7-(1-piperazinyl)-1,8-napthyridine-3-carboxylic acid, 1-cyclopropyl-6-fluoro-1,4-dihydro-4-oxo-7-(piperazinyl)-3-quinolinecarboxylic acid, allylestrertol, cingestol, dehydro-epiandrosteron, dienostrol, diethylstilbestrol, dimethisteron, ethyneron, ethynodiol, estradiol, estron, ethinyl estradiol, ethisteion, lynestrenol, mestranoi, methyl testosterone, norethindron, norgestrel, norvinsteron, oxogeston, quinestrol, testosteron and tigestol; tranquilizers, such as, dofexazepam, hydroxyzin, lorazepam and oxazepam; neuroleptics, such as, acetophenazine, carphenazine, fluphenazine, perphenyzine and piperaetazine; cytostatics, such as, aclarubicin, cytarabinc, decitabine, daunorubicin, dihydro-5-azacytidine, doxortibicin, epirubicin, estramustin, etoposide, tludarabine, gemcitabine, 7-hydroxychlorpromazin, nelarabine, nepIanocin A, pentostatin, podophyllotoxin, tezacitabine, troxacitabine, vinblastin, vincristin, vindesin; hormones and hormone antagonists, such as, buserilin, gonadoliberin, icatibrant and leuprorelin acetate; antihistamines, such as, terphenadine; analgesics, such as, diflunisal, naproxol, paracetamol, salicylamide and salicyclic acid; antibiotics, such as, azidamphcnicol, azithromycin, camptothecin, cefatnandol, chloramphenicol, clarithromycin, clavulanic acid, clindamycin, derriecloeyclin, doxycyclin, erythromycin, gentamyein, imipenem, laramoxef, metronidazole, neomycin, novobiocin, oleandomycin, oxytetracyclin, tetracycline, thiamenicot and tobramycin; antivirals, such as, acyclovir, d4C, ddC, DMDC, Fd4C, FddC, FMAU, FTC, 2′-fluoro-ara-dideoxyinosine, ganciclovir, lamivudine, penciclovir, SddC, stavudine, 5-trifluoromethyl-2′-deoxyuridine, zalcitabine and zidovudine; bisphosphonates, such as, EB-1053, etidronate, ibandronate, olpadronate, residronate, YH-529 and zolendronate; protease inhibitors, such as, ciprokiren, enalkiren, ritonavir, saquinavir and tertakiren; prostaglandins, such as, arbaprostil, carboprost, misoprostil and prostacydin; antidepressives, such as, 8-hydroxychlorimipramine and 2-hydroxyimipramine; antihypertonics, such as, sotarol and fenoldopam; anticholinerogenics, such as, biperidine, procyclidin and trihexyphenidal; antiallergenics, such as, cromolyn; glucocorticoids, such as, betatnethasone, budenosid, chlorprednison, clobetasol, clobetasone, corticosteron, cortisone, cortodexon, dexamethason, flucortolon, fludrocortisone, flumetbasone, flunisolid, fluprednisolon, flurandrenolide, flurandrenolon acetonide, hydrocortisone, meprednisone, methylpresnisolon, paramethasone, prednisolon, prednisol, triamcinolon and triamcinolon acetonide; narcotic agonists and antagonists, such as, apomorphine, buprenorphine, butorphanol, codein, cyclazocin, hydromorphon, ketobemidon, levallorphan, levorphanol, metazocin, morphine, nalbuphin, nahnefen, natoxon, nalorphine, nahrexon, oxycodon, oxymorphon and pentazocin; stimulants such asmazindol and pseudoephidrine; anaesthetics, such as, hydroxydion and propofol; β-receptor blockers, such as, acebutolol, albuterol, alprenolol, atenolol, betazolol, bucindolol, cartelotol, celiproloi, cetamolot, labetatol, levobunelof, metoprolol, metipranolol, nadolol, oxyprenolol, pindolol, propanolol and timotol; α-sympathomimetics, such as, adrenalin, metaraminol, midodrin, norfenefrin, octapamine, oxedrin, oxilofrin, oximetirzolin and phertylefrin; β-sympathomimeties, such as, barnethan, clenbuterol, fenoterol, hexoprenatin, isoprenalin, isoxsuprin, orciprenalin, reproterol, salbutamol and terbutalin; bronchodilators, such as, carbuterol, dyphillin, etophyllin, fcnoterol, pirbuterol, rimiterol and terbutalin; cardiotonics, such as, digitoxin, dobutamin, etilefrin and prenalterol; antimycotics, such as, amphotericin B, chlorphenesin, nystatin and perimycin; anticoagulants, such as, acenocoumarol, dicournarot, phenprocoumon and warfarin; vasodilators, such as, bamethan, dipyrimadol, diprophytlin, isoxsuprin, vincamin and xantinol nicotinate; antihypocholesteremics, such as, compactin, eptastatin, mevinolin and simvastatin; and miscellaneous drugs, such as, bromperidol (antipsychotic), dithranol (psoriasis) ergotamine (migraine) ivermectin (antihelminthic), metronidazole and secnizadole (antiprotozoals), nandrolon (anabolic), propafenon and quinadine (antiarythmics), quetiapine (CNS), serotonin (neurotransmitter) and sitybin (hepatic disturbance).

In some embodiments, an agent can be a nucleic acid, selected from a variety of DNA and RNA based nucleic acids, including fragments and analogues of these, as described herein. A variety of genes for treatment of various conditions have been described, and coding sequences for specific genes of interest can be retrieved from DNA sequence databanks, such as, GenBank or EMBL. For example, polynucleotides for treatment of viral, malignant and inflammatory diseases and conditions, such as, cystic fibrosis, adenosine deaminase deficiency, AIDS, and cancers by administration of tumor suppressor genes, such as, tbr example, APC, DPC4, NF-1, NF-2, MTS1, RB, p53, WT1, BRCA1, BRCA2 and VHL have been described.

In some embodiments, an agent can be, as described herein, a natural or synthetic nucleic acid, or a derivative thereof, single stranded or double stranded, such as, genomic DNA, cDNA, plasmid DNA, DNA vectors, oligonucleotides, or nucleosides, or RNA, including but not limited to sense or antisense RNA, mRNA, siRNA and ribozymes. DNA/RNA hybrids, or peptide nucleic acids (PNA) or derivative thereof. It should be appreciated that such DNA oligonucleotides may be complementary to the coding region, the 3′ untranslated region, or a transcription control sequence of a gene. In some embodiments, the DNA oligonucleotides are modified to increase or decrease biodegradability of the oliganucleotide. In some embodiments, phosphodiester linkages between nucleotides may be replaced with alternative linkages, such as, phosphorothioate linkages or phosphoroamidate linkages.

The polynucleotide may be an antisense oligonucleotide (e.g, DNA and/or RNA) composed of sequences complementary to its target, usually a messenger RNA (mRNA) or an mRNA precursor. The mRNA contains genetic information in the functional, or sense, orientation and binding of the antisense oligonucleotide inactivates the intended mRNA and prevents its translation into protein. Such antisense molecules are determined based on biochemical experiments showing that proteins are translated from specific RNAs and once the sequence of the RNA is known, an antisense molecule that will bind to it through complementary Watson Crick base pairs can be designed. Such antisense molecules typically comprise between 10-30 base pairs (bp). In some embodiments an agent can be a ribozyme or catalytic RNA.

In some embodiments, an agent can be a natural or synthetic peptide or protein, or a derivative thereof. Derivatives of peptides or proteins can be, for example, cyclic, peptides or peptidomimetics, comprising non-natural amino acids and/or non-natural bonds between the individual amino acids. In some embodiments, the agent can be an antibody or antibody fragment, examples of which are well known to those of skill in the art.

The cationic liposomes described herein can also be used to deliver diagnostic agents. Non-limiting examples of diagnostic agents include, for example, enzyme substrates, antibodies, dyes, luminescent compounds and oligonucleotides.

In various exemplary embodiments, an agent can produce a chromogenic, fluorescent, phosphorescent, chemiluminescent or bioluminescent signal. Chemiluminescent compounds include but are not limited to, luminol, isohiminol, theromatic acridinium ester, itaidazole, acridinium salt and oxalate ester. In some embodiments, bioluminescent signals can be produced by biochemical reactions involving luciferin, luciferase and aequorin (UniProtl(B./Swiss-Prot Accession No. P07164).

In some embodiments, cationic liposomes include dyes. In some embodiments, lipophilic fluorescent dyes can be embedded non-covalently within the lipid phase of a liposome to assess the integrity of the liposome or to detect the fusion of the liposome with a membrane. Examples of lipophilic dyes include, but are not limited to, 6-dodecanoyl-2-dimethylaminonaphthalene (LAURDAN) and 6-hexadecanoyl-2-(((2-(trimethylammonium)ethyl)methyl)-amino)naphthalene chloride. (PATMAN) (U.S. Pat. No. 6,569,631). In some embodiments, a membrane impermeable fluorescent dye may be encapsulated in a liposome to act as a tracer to detect fusion and delivery of the liposomal contents into a cell. Examples of such tracers are rhodamine-dextran and fluorescently labeled inulin (U.S. Pat. No. 6,423,547). Lipophilic dyes or tracers can he selected to have spectral characteristics that do not interfere with the detection of the substrates as described herein.

In some embodiments, an agent can be an enzyme substrate. In some embodiments the enzyme substrate provides a detectable signal when modified by an enzyme. In various exemplary embodiments, a detectable signal can be a chromogenic, fluorescent, phosphorescent, chemiluminescent, or bioluminescent. The wavelength of a light signal can be any detectable wavelength, ranging, for example, from ultraviolet, visible, through infrared. Photoluminescence is the process whereby a material can be induced to luminesce when it absorbs electromagnetic radition. Fluorescence and phosphorescence are types of photoluminescence. Chemiluminescence is a process whereby energy can be released from a material in the form of light because of a chemical reaction(s) and requires no light sources for excitation (as is the ease for fluorescence and phosphorescence). An enzyme substrate may be designed and synthesized based upon the specificity of a particular enzyme. Alternatively, substrates may be selected from a wide variety of compounds that are commercially available, or that can be prepared by known techniques. In some embodiments, a substrate can be compatible with a cell such that the cell can remain metabolically active for at least the duration of an assay.

In some embodiments, a substrate can have a leaving group and an indicator group. The leaving group may be selected for removal (e.g, via hydrolytic cleavage) by the enzyme. In some embodiments, the indicator group may be selected or derived from fluorogenic and/or chemiluminescent compounds. Examples of suitable fluorogenic indicator compounds include xanthene compounds such as, for example, rhodamine 110, rhodol, fluorescein, and various substituted derivatives thereof (U.S. Pat. No. 5,871,946), In some embodiments, the indicator group can be selected for its ability to have a first state when joined to the leaving group and a. secon d state when the leaving group is removed from the indicator group. The first state must be detectably different from the second state, however, no particular degree of difference is required. In some embodiments, in the first state an indicator can be less fluorescent than it is in a second state. In some embodiments, an indicator can be fluorescent in both the first and second states, but has an emission profile in the first state that differs from the emission profile in the second state such that one or more emission wavelengths can be monitored in order to detect enzyme activity. In some embodiments, an indicator group can be excitable at a wavelength within the visible range, for example, at wavelength between about 450 to 500 nm. In some embodiments, the indicator group emits in the range of about 480 to about 620 nm, about 500 to about 600 nm, or about 500 to about 550 nm. Auto-fluorescence of many cell types is most prevalent below about 500 nm, and an indicator that emits above this wavelength may be used in some embodiments in order to minimize this potential interference, in some embodiments, a substrate can comprise a dye pair consisting of a donor and an acceptor (i.e., an indicator group) which can be in close proximity in the first state. During an enzymatic reaction, the donor can be cleaved away so that the fluorescence of the indicator drops in the second state. The dye pair can comprise a donor dye which absorbs light at a first wavelength and emits excitation energy in response, and acceptor dye which is capable of absorbing the excitation energy emitted by the donor dye and .fluorescing at a second wavelength in response. A wide variety of dye pairs can be used (U.S. Pat. Nos. 5,800;996, 5,863,727, 5,945,526, 6,130,073 and 6,399,392). In some embodiments, the donor dye may be a member of the xanthene class of dyes, and the acceptor dye may be a member of the xanthene, cyanine, phthlaocyanine, or squaraine class of dyes. In some embodiments, the acceptor has an emission that is greater than about 600 nm or at least about 100 nm greater than the absorbance: maximum of the donor dye. In various exemplary embodiments, the members of a dye pair can be positioned in a substrate such that they can undergo various types of energy transfer as known in the art. In some embodiments, a substrate can comprise a dye pair consisting of an acceptor and a quencher which can be in close proximity in the first state. As a result of an enzymatic reaction, the quencher can no longer absorb the fluorescence of the acceptor so that the fluorescence of the acceptor increases in the second state.

A leaving group can find use for assaying many of the various cellular enzymes as described herein. In non-limiting examples, a leaving group can be selected from amino acids, peptides, saccharides, sulfates, phosphates, esters, phosphate esters, nucleotides, polynucleotides, nucleic acids, pyrimidines, putines, nucleosides, lipids and mixtures thereof. In some embodiments, more than one leaving group can be attached to an indicator group and vice versa. For example, a peptide leaving group and a lipid leaving group can be separately attached to a signal producing compound, such as, rhodamine 110. Other suitable leaving groups can be determined empirically or obtained from the art (Mentlein et al., 1991, Eur. J. Clin. Chem. Clin. Biochem. 29:477 480; Schon etal., 1987, Eur. J. Immunol. 17:1821 1826; FerrerLopez et al., 1992, J. Lab. Clin. Med. 119:231 239; and Royer el al., 1973, J. Biol. Chem. 248:1807 1812).

In some embodiments, luminescent substrates comprising 1,2-dioxetane as an indicator group (such as described in U.S. Pat. Nos. 6,660,529; 6,586,196; 6,514,717; 6,355,441; 6,287,767; and Reissue 36,536) can be used as described herein. Many of these compounds are commercially available (Tropix, Inc., Bedford, Mass.) under the trademarks GALACTO-LIGHT™, GALACTO-LIGHT PLUS™, GALACTO-STAR™, GUS-LIGHT™, PHOSPHA-LIGHT™ and DUAL-LIGHT®. Other suitable substrates include adamantine-dioxetanes, such as, 3-(2′-spiroadamantane)-4-methoxy-(3″-phosphoryloxy)phenyl-1,2-dioxetane disodium salt (AMPPD) and 3-(4-methoxyspiro[1,2-dioxetane-3,2′-tricyclo[3.3.1.1,3,7]decan]-4-yl)phenyl-3-d-galactopyranoside (AMPGD), which are substrates for alkaline phosphatase and p-galactosidase, respectively (e.g, Van Dyke et al., in: Luminescence Biotechnology Instruments and Applications, Van Dyke et at, eds. pages 3-29, CRC Press, 2002). These compounds are available commercially under the trademarks GALACTON®, GLUCON®, GLUCURON® and CSPD®.

In some embodiments, an enzyme substrate can be a β-galactosidyl substituted fluorogenic compound, a β-galactosidyl substituted fluorescein or a substituted derivative thereof. Other examples include 9H-(1,3-dichloro-9,9-dimethylacridin-2-one-7-yl) β-D-galactopyranoside, fluorescein di-β-D-galactoside, 2-nitrophenyl β-D-galactopyranoside, resorufin β-D-galactopyranoside, 6,8-difluoro-4-methylumbelliferyl β-D-galactopyranoside, β-methylumbelliferyl β-D- galactopyranoside, 3-carboxyumbelliferyl β-D-galactopyranoside, 5-chloromethylfluorescein di-β-D-galactopyranoside and 5-(pentalluorobenzoylamino)fluorescein di-β-D-galactopyranoside.

In some embodiments, a substrate can be a 9H-(1,3-dichloro-9,9-dimethylacridin-2-one-7-yl) β-D-galactopyranoside. In some embodiments, the substrate can be fluorescein di-β-galactopyrattoside (catalog no. F-1179, Molecular Probes, Eugene, Oreg.). In some embodiments, a substrate can be 5-bromo-4-chloro-3-indoyl-β-galactopyranoside (3C-gal).

In some embodiments, an substrate can be a P-lactamase substrate. Examples of such substrates include those with a fluorescent donor moiety and an acceptor (e.g, a fluorescence resonance energy transfer (FRET) dye pair), such as, described in U.S. Pat. Nos. 5,955,604 and 6,031,094. Fluorescence energy transfer between the donor and quencher can be monitored as an indicator of β-lactamase activity. β-lactamase substrates have been described which include one or more attached groups (e.g, acetyl, butyryl and acetoxymethyl) which enhances their permeability through cell membranes where the attached group is hydrolytically cleaved by endogenous esterases alter the substrate enters the cell (Klokarnik et al, 1998, Science, 279:84-88; Gao el al., J. Am. Chem. Soc., 2003, 125:11146-11147; and international Publication No. WO 96/30540). In some embodiments, the present methods utilize such substrates. In other embodiments, such substrates are used but lack these attached groups.

β-lactamase substrates include, but are not limited to, 5-Thia-1-azabicyclo[4.2.0]oct-2-ene-2-carboxylicylic acid, 8-oxo-3-[3-[(2-oxo-2H-1-benzopyran-7-yl)oxy]-1-propenyl]-7-[(phenylacetyl)amino]-, (6R,71t)-(9CI, CA Registry No. 609812-88-6)); 5-Thia-1-azabicyclo[4.2.0]oct-2-cnc-2-carboxylic acid, 8-oxo-3-[3-[(2-oxo-2H-1-benzopyran-7-yl)oxy]-1-propenyl]-7-[(phenylacetyl)amino]-, 5-oxide, (6R,7R)-(9CI) (CC2, (CA Reg. No. 609812-89-7)); 5-Thia-1-azabicyclo[4.2.0]oct-2-ene-2-carboxylic acid, 8-oxo-3-[(1Z)-3-[(3-oxo-3H-phenoxazin-7-yl)oxy]-1--propenyl]-7-[(2-thienylacetyl)amino]-, 5-oxide, (6R,7R)-(9CI), (CA Registry No 452280-30-7)); 5-Thia-1-azabicyclo[4.2.0]oct-2-ene-2-carboxylic acid, 7-[[[[(6-chloro-7-hydroxy-2-oxo-2H-1-benzopyran-3-yl)carbonyl]amino]acetyl]amino]-3-[[(3′,6″-dihydroxy-3-oxospiro[isobenzofuran-1(3H),9′-[9H]xanthen]-5-yl)thio]methyl]-8-oxo-, (6R,7R)-(9CI) (CCF2, (CA Registry No. 1873736-52-9)), 5-Thia-1-azabicyclo[4.2.0]oet-2-ene-2-carboxylic acid, 8-oxo-3-[3-[(3-oxo-3H-phenoxazin-7-yl)oxy]-1-propenyl]-7-[(2-thienylacetyl)amino]-, (acetyloxy)methyl ester, 5-oxide, (6R,7R)-(9CI) (CR2/AM, (CA Regsitry No. 452280-31-8)); 5-Thia-1-azabicyclo[4.2.0]oct-2-ene-2-carboxylic acid, 7-[[[[[7-[(acety loxy)methoxy]-6-chloro-2-oxo-2H-1-1-ben zopyran-3-yl]carbonyl]amino]acetyl]amino]-3-[[[3′,6′-bis(acetyloxy)-3-oxospiro[isobenzofuran-1(3H),9′-[9H-xanthen]-5-yl]thio]methyl]-8-oxo-, (acetyloxy)methyl ester, (6R,7R)-(9CI) (CCF2/AM, (CA Registry No 1837366-66-5), and/or mixtures thereof.

In some embodiments, a substrate can he a substrate of a luciferase enzyme. Examples, include varglin luciferin (Catalog No. NF-CV-HBR, ‘Nanolight Technology, Pinetop, Ariz.), coelenterazine (Catalog No. NF-CTZ-FB, Nanolight Technology, Pinetop, Ariz.; and Catalog No. E2810 and Part No. TM055, Promega, Madison, Wis.), firefly luciferin (D-(−)-2-(6’-hydroxy-2′-benzothiazolyl)thiazoline-4-carboxylic acid (available from Pierce Biotechnology, Molecular Probes, and Nanolight), cyprinda luciferin (Catalog No. NF-CV-HBR, Nanolight Technology), bacterial luciferin, clinoflagellate luciferin and/or mixtures thereof.

In some embodiments, a substrate can be a substrate of various cytochrome P450 isozymes. Examples include luciferin 6′ chloroethyl ether (luciferin-CEE, Catalog No. V8751, Promega, Madison, Wis.), luciferin 6′ methyl ether (luciferin-ME, Catalog No. V8771, Promega, Madaison, Wis.), 6′-deoxyluciferin (luciferin H, Catalog No. V8791, Promega, Madison, Wis.), luciferin 6′ benzyl ether (luciferin-BE, Catalog No. V8801, Promega, Madison, Wis.) and/or mixtures thereof.

In various exemplary embodiments, a substrate can be a substrate of β-glucuronidase, carhoxylesterasc, lipases, phospholipases, sulphatases, ureases, peptidases, sulfatases, thioesterases, and proteases. lii sonic embodiments, the enzyme is a hydrolytic enzyme. Non-limiting examples of hydrolytic enzymes comprise alkaline and acid phosphatases, esterases, decarboxylases, phospholipase D, P-xylosidase, β-fucosidase, thioglucosidase, α-galactosidase, α-glucosidase, β-glucosidase, β-glucuronidase, α-mannosidase, β-mannosidase, β-fructofuranosidase, β-glucosidase, and trypsin. Other examples of enzymes comprise hydrolases, oxidoreductases, saccharidases, β-glucosidase, β-lactamases, β-glucuronidase, α-galactosidase, β-hexosaminidase, cholesterol esterase, nucleases, arylsulfatase, phospholipase, caspase 1, caspase 2, caspase 3, caspase 4, caspase 5, caspase 6, caspase 7, caspase 8, caspase 9, caspase 10, caspase 11, caspase 12, caspase 13, caspase 14, luciferases, and phosphatase. Specific examples of enzymes comprise E. coli β-glucosidase, E. coli TME-1 β-lactamase, glutathione-S-transferase, chloramphenicol acetyltransferase (CAT), uricase, secreted form of human placental alkaline phosphatase (SEAP), dihydrofolate reductase (DHFR), protein kinase A (PKA), protein kinase (PKC) isozymes (e.g, PKCα, PKCβ and PKCγ), fatty acid synthase, cysteine protease, and phospholipase A2.

In various exemplary embodiments, a substrate can be a substrate of phosphorylase kinase (Phk) cyclin-dependent kinase-2 (cdk2), ERK and extracellular-regulated kinase-2 (ERK2), Ca2+/calmodulin-dependent protein kinase I (CAMKI), Ca2+/calmodulin-dependent protein kinase II (CAMKII), cellular form of Rous sarcoma virus transforming agent (c-SRC), transforming agent of Fujinami sarcoma virus (v-FPs), C-terminal Src kinase (Csk), Insulin receptor kinase (InRK), EGF receptor, Src kinase (SRC). RAC-beta serine/threonine-protein kinase (Akt), Extracellular signal-regulated kinase I (MAP kinase 1)(Erk1), MAP kinase-activated protein kinase 2 (MAPKAP), MEK, JNK, Ras, Serine/threonine-protein kinase Nek2, tyrosine kinase Ab1, Proto-oncogene tyrosine-protein kinase YES and LCK, Tyrosine-protein kinase LYN and BTK, glycogen synthase kinase-3 (GK3), casein kinase I, and casein kinase II.

Non-limiting examples of enzymes substrates for enzymes include the following: estrogen sulfotransferase (SULT 1E); estrone sulfatase (E.C. 3.1.6.2.) as assayed using a substrate, such as, substrate 3,4-benzocoumarin-7-O-sulfate (Bilban, et al., 2000, Bioorganic and Medicinal Chemistry Letters 10:967-969; farnesyl:protein transferase which can be detected using a substrate, such as, N-dansyl-GCVLS (Pompliano, et al., 1992, J. Am. Chem. Soc. 114:7945-7946); sialyl transferase (E.C. 2.4.99.1) which can utilize a glycosyl donor, such as, Nap-CMP-NANA or a glycosyl acceptor, such as, LacNAc-Dan (Washiya, et al., 2000, Analytical Biochemistry 283:39-48); histone deacetylase which can be detected using a substrate, such as, MAL (Sigma catalog no. H 9660); caspase 8 which can be monitored using a substrate, such as, Z-IETD-R110 (Molecular Probes catalog no. A-22125); and selected cytochrome P450 isozymes which oxidize substrates, such as, ethoxyresorufin (Sigma catalog no. CYTO-1A). Further non-limiting examples of enzymes include: protein kinases, estrogen sulfotransferases, carbohydrate sulfotransferases, tyrosylprotein sulfotransferases, farnesyl transferases, COX-1, 2, dihydrofolate reductase, aromatase, alcohol dehydrogenase, acetylcholinesterase, sialyl transferase, adenylyl cyclase, inositol phosphoceramide (IPC) synthase, glycosyl transferases, lanosterol 14α-demethylase, type 2 fatty acid synthase, thymidylate synthase, geranylgeranyl transferase, methionine synthase, serine hydroxymethyltransferase, HMG-CoA reductase, histone acetyltransferase, histone deacetylase, cyclic nucleotide phosphodiesterases, phosphoinositide 3 kinase, 17β-hydroxysteroid dehydrogenase, topoisomerase, telomerase, squalene synthase, palmatoyl transferase, myristoyl transferase.

In some embodiments, phosphorylation activity of an enzyme can be monitored. For example, a fluorescent-labeled oligopeptide (DACM-CLRRASLK-fluorescein), containing a consensus amino acid sequence (RRXSL) of cyclic AMP (cAMP) dependent protein kinase A (PKA) substrate-proteins (Ohuchi et al., 2000, Analyst 125:1905-1907; Ohuchi et al., 2001, Analytical Sci. (supp.) 17:i1465-i1467), may be used as a substrate in the present methods. The phosphorylation of the serine residue in the substrate causes a change in fluorescent intensity. Other suitable substrates include one or more members of the library of fluorescently-labeled PKC substrates (Yeh et al., J. Biol. Chem. 277:11527-11532) and fluorescent peptide substrates for PKC and PKA which contain a kinase sensing motif (Shults et al., 2003, J. Am. Chem. Soc. 125:14248-14249). Non-limiting examples of suitable peptide substrates include those described in Shults et al., 2003, J. Am. Chem. Soc. 125:14248-14249.

In some embodiments, a diagnostic agent is not at least substantially cell membrane permeable or requires invasive delivery methods, such as, hypotonic shock, electroporation, microinjection etc. In some embodiments, the diagnostic agent can be a fluorescein digalactoside (Molecular Probes catalog no. F1179); DDAO phosphate (Molecular Probes catalog no. D-6487); Fluo-3 (Molecular Probes catalog no. F-1240); Alexa Fluor 488 phalloidin (Molecular Probes catalog no. A12379); dextran, or Alexa Fluor 488 (Molecular Probes catalog no. D-22910). While the agent Fluo-3 (F-1240) can be prepared with biodegradable AM protecting groups (Molecular Probes catalog no Fluo-3, AM, F-1241) which make it cell membrane permeable the AM is enzymatically cleaved one inside a cell. The cationic liposomes described herein can allow for the delivery of Fluo-3 without the chemical derivatization.

In some embodiments, a compound or agent can affect or modify (e.g, increase or decrease) expression or activity of a target protein. Therefore, in various exemplary embodiments an agent can modify or affect transcription, translation, post-translational modification, and/or activity of a protein. In various exemplary embodiments, a compound can be capable of silencing a target protein.

In various exemplary embodiments, a compound/agent can be capable of silencing a target protein. “Silencing a target protein” as used herein refers to inhibition of the target protein at the level of transcription, translation., post-translational modification, and/or the protein itself. In some embodiments, a compound can be capable of inhibiting transcription of DNA encoding the target protein. In some embodiments a compound can be capable of inhibiting translation of an mRNA encoding the target protein. In some embodiments, a compound can be capable of inhibition of mRNA processing. In some embodiments, a compound can be capable of inhibiting post-translational processing of a target protein. In some embodiments, a compound can be capable of inhibition one or more target protein activities or functions or metabolism. “Apparent activity” as used herein refers to the activity of a target protein that can be measured a result of modifying or affecting the concentration of a target protein, such as in an intracellular environment, by various methods, including but not limited to, modifying transcription, translation, or post-translational processing of the protein, as described herein.

In various exemplary embodiments, a compound comprises an oligonucleotide. For example an oligonucleotide can modulate the function of nucleic acid molecules encoding the target protein, which ultimately modulates the amount of target protein produced. This can be accomplished by providing an oligonucleotide which specifically hybridizes with one or more nucleic acids encoding the target protein. As used herein, the terms “target nucleic acid” and “nucleic acid encoding target protein” encompass DNA encoding target protein, RNA (including pre-mRNA and mRNA) transcribed from such DNA, and also cDNA derived from such RNA. The specific hybridization of an oligomeric compound with its target nucleic acid interferes with the normal function of the nucleic acid. The modulation of a function of a target nucleic acid by compounds which specifically hybridize to it is generally referred to as “antisense”. Thus, in some embodiments, the oligonucleotide can be an antisense compound.

The functions of DNA that can be inhibited include replication and transcription. The functions of RNA to be interfered with can include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function can decrease expression of the target protein.

The composition of the oligonucleotide compound depends on the choice of target protein to be silenced. The process usually begins with choosing a target protein of interest and the identification of its nucleic acid sequence whose function is to he modulated. This may be, for example, a cellular gene (or mRNA transcribed from the gene) whose expression is associated: with a particular disorder or disease state, or a nucleic acid molecule from an infectious agent. The targeting process also includes determination of a site or sites within this gene for the antisense interaction to occur such that the desired effect, e.g., detection or modulation of expression of the protein, will result. In various exemplary embodiments, the intragenic site include the region encompassing the translation initiation or termination codon of the open reading frame (ORE) and the ORF of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codons having the .RNA sequence 5′-GUG, 5′-UUG or 5′-CUG, and 5′-AUA, 5′-ACG and 5′-CUG have, been shown to function in vivo. Thus, the terms “translation initiation codon” and “start codon” can encompass many codon sequences, even though the initiator amino acid in each instance is typically methionine (in eukaryotes) or fortnylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the present disclosure, “start codon” and “translation initiation codon” refer to the codon or codons that are used in vivo to initiate translation of an mRNA molecule transcribed from a gene encoding a target protein, regardless of the sequence(s) of such codons.

It is also known in the art that a translation termination codon for “stop codon”) of a gene may have one of three sequences, i.e., 5′-UAA, 5′-UAG and S′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic inRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be target regions, and are useful in situations where aberrant splicing is implicated in disease, or where an overproduction of a particular :mRNA splice product is implicated in disease. Aberrant fusion junctions due to rearrangements or deletions can be targeted. It has also been found that introits can also be effective target regions for antisense compounds targeted, for example, to DNA or pre-mRNA.

Once one or more target sites have been identified, oligonucleotides can be chosen which are sufficiently complementary to the target, i.e., hybridize sufficiently well and with sufficient specificity, to give the desired effect.

In the context of this disclosure, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of compIementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired.

In the context of this disclosure, the term “oligonucleotide” refers to an oligomer or polymer of deoxyribonucleic acid (DNA), or ribonucleic acid (RNA), oligonucleotide analogs, and oligonucleotide mimetics. The oligonucleotide can include naturally-occurring nucleobases, sugars and covalent internucicoside (backbone) linkages as well as non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

In some embodiments, an. oligonucleotide can comprise from about 8 to about 30 t⁻tticleobases. In some embodiments the oligonucleotide comprising from about 8 to about 30 nucleobases (i.e., front about 8 to about 30 linked nucleosides). in some embodiments oligonucleotides comprise at least an 8-nucleobase portion of a sequence of the compound which inhibits expression of target protein. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be finked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, in some embodiments open linear structures are can be used. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

In some embodiments, an oligonucleotide comprises a modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In some embodiments, antisense compounds comprises a modified oligonucleotide backbones, fur example, phosphorothioates, chiral phosphorothioates, phosphorodititioates, phosphotriesters, am inoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2%5′ linked analogs of these, and those having inverted polarity wherein the adjacent pairs of nucleoside units are linked 3′-5′ to 5-3′ or 2′-5′ to 5′-2′. Various salts, mixed salts and free acid forms are also included.

In some embodiments, the oligonucleotide comprises a modified oligonucleotide backbone that does not include a phosphorus atom but has a backbone that is formed by short chain alkyl or cycloalkyl internucleoside linkages, mixed heteroatom and alkyl or cycloalkyl internucleoside linkages, or one or more short chain heteroatomic or heterocyclic intemucleosicle linkages. These include those having morpholino linkages (formed in part from the sugar portion of a nucleoside); siloxan.e backbones; sulfide, sulfoxide and sulfone backbones; formacetyl and thioformacetyl backbones; methylene forrnacetyl and thiofbrmacetyl backbones; alkene containing backbones; sulfamate backbones; methyleneirnino and m.ethylenehydrazino backbones; sulfonate and sulfonamide backbones; amide backbones; and others having mixed N, O, S and CH₂ component parts.

In various exemplary embodiments, a compound can be an oligonucleotide mimetic. In some embodiments the oligonucleotide mimetic, both the sugar and the internucleoside linkage, i.e., the backbone, of the nucleotide units can be replaced with novel groups. The base units are maintained for hybridization with an appropriate nucleic acid target compound. In some embodiments the oligonucleotide mimetic can be a peptide nucleic acid (PNA). In PNA, the sugar-backbone of an oligonucleotide is replaced with an amide containing backbone, such as, an aminoethylglyeine backbone. PNA differs from DNA in that the negatively-charged ribose-phosphate backbone of the latter is replaced by its neutral peptide counterpart, for example, glycine. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference.

In various embodiments, a compound can be capable of silencing a target protein by inhibiting a post-translational modification of the target protein. For example, in some embodiments a compound is capable of inhibiting the phosphorylation and/or dephosphorylation of the target protein. in some embodiments, a compound is capable of inhibiting the glycosylation of the target protein. In some embodiments, a compound is capable of inhibiting the prenylation of the target protein. In some embodiments, a compound is capable of inhibiting the rnyristoylation of the target protein. In some embodiments, a compound is capable of affecting the tertiary structure folding of the target protein.

In various embodiments, a compound can be capable of controlling the apparent activity of a target protein via Ca ²⁺, adenosine triphosphate (ATP), guanosine triphosphate (GTP) diacylglycerols (DAG), inositol 1,4,5-tris-phosphate (IP₃), protein:protein interaction or through a signal transduction cascade, For example, the target protein can be activated or deactivated by a protein:protein interaction. If the protein partner is deactivating and the compound acts up the protein partner then the apparent activity of the target protein will be increased. Conversely, if the protein partner is activating and the compound acts up the protein partner then the apparent activity of the target protein will be decreased.

In some embodiments, such as, when the target protein is an enzyme, a compound can be capable of controlling the activity of a target protein. In some embodiments, a compound can be capable of silencing a target protein by directly inhibiting the target protein with a modulator, such as, enzyme inhibitor.

As mentioned above a target protein can be any protein of interest. In some embodiments a target protein can be an enzyme. Non-limiting examples of target proteins include, but not limited to ATPases, adapter molecules, adenylate cyclases, adhesion molecules, alkaline phosphatases, aminopeptidases, anchor proteins, 9 cell antigen receptors, CD antigens, calcium binding proteins, cell cycle control proteins, cell junction proteins, cell surface receptors, chaperones, chaperonins, chemokines, coagulation factors, complement proteins, complement receptors, cysteine proteases, cytokines, cytokine receptors, cytoskeletal associated proteins, cyteskeletal proteins, DNA binding proteins, DNA ligases, DNA methyltransferases, DNA polymerases, DNA repair proteins, defensins, deo.xyribonucleases, dual specificity kinases, dual specificity phosphatases, acid phosphatases, acyltransferases, adenosyltransferases, aldolases, amidinotransferases, aminomethyl transferases, aminotransferases, amylases, carbamoyitranskrases, carbonic anhydrases, carboxylases, catalases, CoA transferases, cyclotransferases, deacetylases, deaminases, decarboxylases, dehydratases, dehydrogenases, dephosphorylases, epimertases, esterases, fucosyltransferases, galactosidases, galactosyltransferases, glitcosaminyltransferases, glucosidases, glucuronidases, glutamyltransferases, glutathione transferases, glycosidases, glycosylases, .glycosyltransferases, guanyl cyclases, hydratases, hydrolases, hydroxylases, isomerases, ligases, lipases, lyases, mannosyltransferases, methylamine transferases, methyltransferases, mutases, NADases, nucleotidyltransfrrases, oxidases, oxidorechictases, oxygenases, palmitoyltransferases, peroxidases, pbosphodiesterases, phosphohydrolases, phospholipases, phosphoribosyltransferases, phosphorylases, phospbotransferases, prenyltransferases, racemases, reductases, ribosyltransferases, sialyltransferases, sugar phosphotransferases, sulphatases, sulphohydrolases, sulphotransferases, superoxide dismutases, survivins, synthetases, topoisomerases, transaldolases, transaminases, transferases, transketolases, translocases, extraeellular ligand gated channels, extracellular matrix proteins, G proteins, G protein coupled receptors, GTPases, GTPase activating proteins, growth factors, guanine nueleotide exchange factors, guanylate cyclases, heat shock proteins, immunoglobulins, integral membrane proteins, intercellular channels, intracellular ligand gated cbaanels, inward rectifier channels, ion channels, lipid kinases, lipid phosphatases, MEC complex proteins, membrane bound ligands, membrane transport proteins, metallo proteases, motor proteins, neuraminidases, nuclear receptors, peptide hormones, protease inhibitors, protemes, RNA binding proteins. RNA methyliransferases, RNA polymerases, receptor serine/threonine kinases, receptor tyrosine kinases, receptor tyrosine phosphatases, reverse trartscriptases, riboriucleases, ribonucleoproteins, ribosomal subunits, secreted polypeptides, serine proteases, serine/threonine kinases, serine/threonine phosphatases, storage proteins, structural proteins, T cell antigen receptors, transcription factors, transcription regulatory proteins, translation regulatory proteins, transport/cargo proteins, tyrosine kinases, tyrosine phosphatases, ubiquitin proteasome system proteins, voltage gated channel proteins, and water channel proteins.

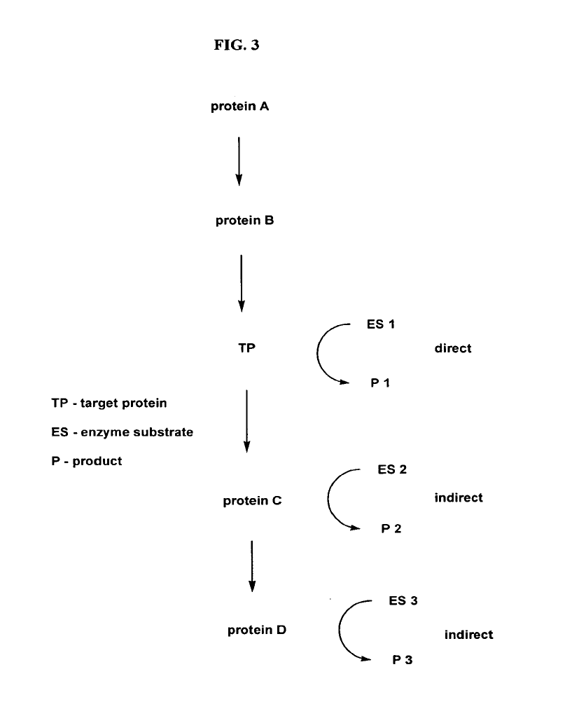

In some embodiments, a liposome, such as a cationic liposome as described herein, comprising a compound capabale of silencing a target protein can further comprise an enzyme substrate capable of producing a detectable signal. A wide variety of enzymes substrates can be used., including but not limited to enzyme substrates that are capable⁻ of producing a detectable signal when modified by an enzyme, as described herein. In some embodiments, an enzyme substrate can be capable of producing a detectable when modified by a readout protein. In some embodiments a readout protein can be a target protein. For example, FIG. 3 shows an enzyme substrate ES.I capable of producing a detectable signal PI when modified by the target protein TP. In some embodiments, a readout protein can be “downstream” of a target protein, for example in a signal transduction cascade. For example, FIG. 3 shows an enzyme substrate ES 2 capable of producing a detectable signal P2 when modified by the readout protein protein C and where enzyme substrate ES 3 capable of producing a detectable signal P3 when modified by the readout protein protein D.

The activity of a readout protein can he positively or negatively coupled to a target protein. In some embodiments, a readout protein can be coupled to a target protein through a protein:protein interaction. In some embodiments, a readout protein can he coupled to the target protein through a signal transduction cascade.

An enzyme substrate can be designed and synthesized based upon the specificity of a particular enzyme. Alternatively, an enzyme substrate can be selected from a wide variety of substrates that are commercially available, or that can be prepared by known techniques. In some embodiments, the enzyme substrate can be compatible with the cell such that the cell will remain metabolically active for at least the duration of the assay.

5.3 Methods

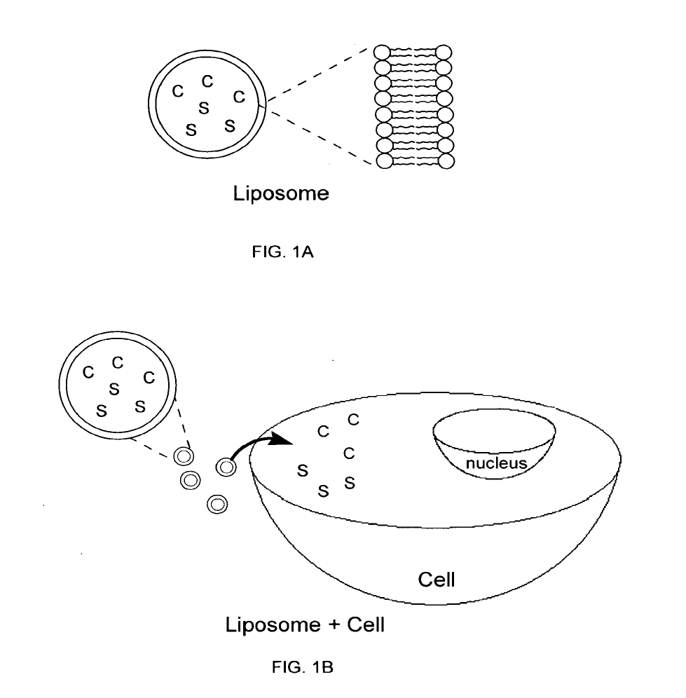

The present disclosure is also directed to a method of delivering one or more compounds or agents to a cell with a cationic liposome. A cell can be contacted with the cationic liposome encapsulating or complexing a compound or agent, such as, those described herein. In some embodiments, a liposome can contain at least two or more compounds. In some embodiments, liposome can comprise (i) a compound capable of silencing a target protein and (ii) an enzyme substrate. FIG. 1A illustrates an exemplary embodiment of a liposome comprising a compound C capable of silencing a target protein and a enzyme substrate S. FIG. 1B illustrates an exemplary embodiment of a liposome delivering the compound C and a enzyme substrate S into a cell. In some embodiments, a liposome can contain two or more enzyme substrates, wherein the substrates are capable of producing distinguishable signals, such as, two distinguishable fluorescent signals (U.S. Pat. No. 5,863,727). in some embodiments, methods for detecting or analyzing a. target protein in a living cell and for determining one or more enzymatic pathways and/or signal transduction pathways in which a target protein is a component are provided.

Cell types which may be used with the liposomes described herein include eukaryotic (e.g, animals, plants, yeast, fungi) and bacterial cells. Viable cells that can be used include fresh cells isolated from a living organism, cells grown or cultured in vitro, or cells reconstituted from frozen or freeze-dried preparations. Cells having a cell wall can be used after appropriate measures are taken to remove the cell wall (Constabel, 1982, in “Plant Tissue Culture Methods” pp. 38-48, NRCC No. 19876, Nat. Res. Council of Canada, Saskatoon.). Further examples of cells, which may be used with the liposomes described herein are primary or established cell lines and other types of embryonic, neonatal or adult cells, or transformed cells (for example, spontaneously- or virally-transfbrined). These include, but are not limited to fibroblasts, macrophages, myoblasts, osteoclasts, osteoclasts, hematopoietic cells, neurons, glial cells primary B- and T-cells, B- and T-cell lines, chondrocytes, keratinocytes, adipocytes and hepatocytes.

Cell lines which can be used with the liposomes described herein include, but are not limited to, those available from cell repositories, such as, the American Type Culture Collection (www.atcc.org), the World Data Center on Microorganisms (wdem.nig.ac.jp), European Collection of Animal Cell Culture (www.ecacc.org) and the Japanese Cancer Research Resources Bank (cellbank.nihs.go.jp). These cell lines include, but are not limited to, Jurkat, 293, 293Tet-Off, CHO, CHO-AA8 Tet-Off, MCF7, MCF7 Tet-Off, LNCap, T-5, BSC-1, BHK-21 Phinx-A, 3T3, HeLa, psi Bagα, PC3, DU145, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, CRL-1573, 3T3L1, Cama-1, J774A.1, HeLa 229, PT-67, Cos7, OST7, HeLa-S, THP-1, Jurkat, GHK-21, CHO-K1, COS7, COS, HepG2, PC12, 293, A431, A459, 1B, L929 and NXA cell lines. Additional cell lines for use with the liposomes described herein can be obtained, for example, from cell line providers, such as, CIoneties Corporation (Walkersville, Md.).

In some embodiments, a cell suspension or attached cells are admixed with a suspension of cationic liposomes encapsulating or complexing an agent as described herein. The admixture is maintained for a time period and under physiological reaction conditions sufficient for the agent to enter the cells.

Essentially any medium that is compatible with the cell line and experimental conditions may be used with the liposomes and methods described herein. For example, a variety of cell culture media are described in “The Handbook of Microbiological Media” (Atlas and Parks, eds.) (1993, CRC Press, Boca Raton, Fla.). References describing the techniques involved in bacterial and animal cell culture include Sambrook et al., Molecular Cloning-A Laboratory Manual (2nd Ed.), Vol. 1-3 (1989, Cold Spring Harbor Laboratory, Cold Spring Harbor, N.Y.); Current Protocols in Molecular Biology, F. M. Ausubel et al., eds., Current Protocols, (a joint venture between Greene Publishing Associates, Inc. and John Wiley & Sons, Inc., supplemented through 2000); Preshney, Culture of Animal Cells, a Manual of Basic Technique, third edition (1994, Wiley-Liss, New York) and the references cited therein; Humasort, Animal Tissue Techniques, fourth edition (1979, W. H. Freeman and Company, New York); and Ricciardelli, et al., 1989, In Vitro Cell Dev, Biol. 25:1016-1024. Information regarding plant cell culture can be found in Plant Cell and Tissue Culture in Liquid Systems, by Payne el al, (1992, John Wiley & Sons, Inc. New York, N.Y.); Plant Cell, Tissue and Organ Culture: Fundamental Methods by Garnborg and Phillips, eds. (1995, Springer Lab Manual, Springer-Verlag, Berlin), and is also available in commercial literature, such as, the Life Science Research Cell Culture Catalogue (1998) from Sigma-Aldrich, Inc (St Louis, Mo.) (Sigma-LSRCCC) and the Plant Culture Catalogue and supplement (1997) also from Sigma-Aldrich (Sigma-PCCS). Particular non-limiting examples of suitable media include conventional cell culture media. Such media are widely available (e.g, Sigma-Aldrich) and include Earle's Balanced Salts, Hanks' Balanced Salts, Tyrode's Salts and other salt mixtures.

Media containing serum can be used with the disclosed liposomes and methods disclosed herein. Non-limiting examples of suitable serum (all of which are commercially available, e.g., Sigma-Aldrich) include fetal bovine serum (PBS), bovine serum, calf serum, newborn calf serum, goat serum, horse serum, human serum, chicken serum, porcine serum, sheep serum, serum replacements, embryonic fluid, and rabbit serum. In some embodiments, media comprising PBS in the range of about 2% to about 10% (v/v), in the range of about 4% to about 7%, and at a concentration of about 5%, can be used.

Suitable media can include an aqueous medium having an osmolality, tonicity, pH value and ionic composition that supports and maintains cell viability. Exemplary media include, but are not limited to normal saline, Ringer's solutions and commercially available cell culture media, such as, minimum essential medium (MEM), RPMI, Dulbecco's and Eagle's medium. One example of a suitable medium is buffered saline consisting of 4% v/v fetal calf serum, 10 mM Hepes, pH 7.2 at a temperature in the range of about 20° C.-37° C.

Methods described herein can be carried out under various reactions conditions. The reaction conditions can he selected to reduce or minimize adverse effects on the ecil, and to not significantly interfere with the interaction of the liposomes with the cells or the detection of a light signal. in some embodiments, the conditions are essentially the same as those conventionally used to maintain viable cultures of cells, Reaction conditions can include selected values of temperature, pH vadue, osmoiality, tonicity and the like. The pH is between about 6.0 to about 8.5 and, in certain cases from a about 6.5 to about 7.5. The osmolality is between about 200 milliosmols per liter (mOsni) and about 500 mOsm and, in some embodiments, from about 250 mOsm to about 350 mOstn. Tonicity can be maintained isotonic to the cells being used. The temperature during detection of enzyme activity may be maintained at any temperature compatible with the cells. In some embodiments, the temperature is maintained at or near the membrane freezing point of the cell. In some embodiments, the temperature can be above the membrane freezing point of the cell. In various exemplary embodiments, the temperature can be at least about 4° C., about 10° C., about 15° C., about 20° C., about 30° C., about 37° C., about 40° C., or about 42° C., In some embodiments, the temperature can be between about 10° C. and about 50° C. and, in some embodiments between about 20° C. and about 40° C.

ln one aspect, methods of detecting the apparent activity of a target protein are disclosed. In some embodiments, the method comprises contacting a cell with a compound capable of silencing a target protein and an enzyme substaate capable of producing a detectable signal when modified by a readout protein, where a change of detectable signal indicates an apparent activity of a target protein, In some embodiments the method comprises contacting a cell with a liposome comprising (i) a compound capable of silencing a target protein and (ii) an enzyme substrate capable of producing a detectable signal when modified by a readout protein, where a change of detectable signal indicates an apparent activity of a target protein.

In another aspect, methods of inhibiting the expression of a target protein and detecting and/or monitoring the inhibition in a live cell are disclosed. In some embodiments, a cell can be contacted with a compound capable af silencing a target protein and an enzyme substrate capable of producing a detectable signal when modified by a readout protein, In some embodiments, a cell can be contacted with a liposome comprising a compound capable of silencing a target protein and an enzyme substrate capable of producing a detectable signal when modified by a readout protein. A change in detectable signal indicates an inhibition of expression of the target protein in the cell. In some embodiments, the amount of detectable signal can be compared with a control. The control can be the amount of detectable signal produced by contacting a live cell with the enzyme substrate.

In another aspect, methods of identifying a target protein associated with a signal transduction pathway of interest in a live cell are disclosed. In some embodiments, the method comprises contacting a cell with a compound capable of silencing a target protein and an enzyme substrate capable of producing a detectable signal when modified by a readout protein. In some embodiments, the method comprises contacting said cell with a liposome comprising (i) a compound capable of silencing a target protein and (ii) an enzyme substrate capable of producing a detectable signal when modified by a readout protein. The cell can be contacted with an agonist of the signal transduction pathway. A change of detectable signal indicates that the target protein is associated with the signal transduction pathway of interest. In some embodiments, the detectable signal can be compared with a control. The control can be the amount of detectable signal produced by contacting a live cell with the enzyme substrate.

A wide variety of signal transduction pathways and associated proteins can be investigated. Non-limiting examples of signal transduction pathways and proteins include AKT signaling, also known as protein kinase B (PKB), a serirte/threortine kinase, an enzyme in several signal transduction pathways involved in cell proliferation, apoptosis, aneiogenesis, and diabetes; signal transduction pathways of Alzheimer's disease including the protein β-amyloids, tau proteins, secretases, presenelins, glycogen synthase kinase; vasculogenesis and tumor angiogenesis signaling pathways; apoptosis signaling pathways, such as, mitochondrial apoptosis and caspase mediated apoptosis; Mitogen-activated protein (MAP) kinases mediated signal transduction from growth hormones, heat shock, UV radiation, osmolarity or cytokines; Insulin and IGF-1 activated mitogenic MAP kinase pathway in acquired insulin resistance associated with type 2 diabetes; ERK1/ERK2 MAP kinases signaling pathways; JNK/SAPK (c-Jun kinase/stress activated protein kinase) MAP kinase cascade; p38 kinase cascades; eukaryotic nuclear factor kB (NF-kB) signal transduction pathways; nitric oxide signaling pathway; p53 signaling; protein kinase activation, such as, protein kinase C (PKC) involved in signal transduction associated with cell proliferation, differentiation, and apoptosis; protein tyrosine kinases (PTKs) pathways in cell proliferation, differentiation, metabolism, migration, and survival; G-protein coupled receptor pathways, insulin pathways; VEGF pathways; and the ubiquitin-protcosome degradation pathway, and any combinations thereof. Agonists of various pathways are known in the art.

In some embodiments the apparent activity of a target protein can be measured indirectly in a live cell. For example, a target protein can be survivin. Survivin is a member of the inhibitor of apoptosis protein (IAP) family. lAPs have been reported to directly inhibit active caspase-3 and 7 which execute the apoptotic program by cleaving numerous cellular proteins. Survival binds specifically to the effector cell death proteases (i.e. caspase 3 and 7) and inhibits caspase activity and cell death in cells exposed to apoptotic stimuli. Thus, the inhibition of survivin expression can be detected with an enzyme substrate for caspase 3 which is capable of producing a detectable signal when modified by caspase 3 the activity of which is induced by the inhibition of the expression of survivin.

In some embodiments, the apparent activity of a target protein can be measured indirectly in a live cell, in some embodiments the enzyme substrate is capable of producing a detectable signal when modified by an enzymatic activity downstream from the target protein in a signal transduction pathway cascade. For example, the inhibition of expression of a G-protein coupled receptor can be detected with enzyme substrate for PKC which is capable of producing a detectable signal when modified by PKC.

In some embodiments, following contact of a compound and an enzyme substrate with the cell, a light detectable signal (e.g, a fluorescent or a chemiluminescent signal) in the cell is measured. A change (e.g, an increase or a decrease as compared with a control cell) in the light signal is indicative of enzyme activity and inhibition of expression of a target protein. The light signal may he detected at one or more discrete time points following contact or, alternatively, the light signal may be detected substantially continuously as a function of time. Changes in light signal may be due to the activity of a single enzyme, or may be due to the cumulative activities of several different enzymes that have the same observable activity. In some eases, in can be desirable to selectively inhibit a particular enzyme.

In some embodiments, following contact of a compound and an enzyme substrate with the cell, a light detectable signal in the cell is measured. In some embodiments, the absence of detectable signal indicates an inhibition of expression of the target protein. In some embodiments, a change in the amount of detectable signal can be compared with a control cell to determine the inhibition of expression of a target protein. The control can be the amount of detectable signal produced by contacting a cell with a liposome composition comprising an enzyme substrate but the target protein silencing compound.

Iin the methods described herein, a compound can be transferred into the cell in an amount suitable to change the apparent activity of a target protein. No particular concentration of compound is required as long as the change in the apparent activity of a target protein can be detected. A suitable concentration of compound can be determined empirically, and cells that are known to possess the protein under study can be used as a basis for selecting such concentrations. Liposomes can be prepared using various concentrations of the compound and/or various amounts of liposomes can be used.

In the methods described herein, a substrate can be transferred into the cell in an amount suitable for generating a light detectable signal. No particular concentration of substrate is required as long as a signal can be detected. A suitable substrate concentration can be determined empirically, and cells that are known to possess the enzyme under study can be used as a basis for selecting such concentrations. Liposomes can be prepared using various concentrations of substrate and/or various amounts of liposomes can be used.

When a liposomal preparation is contacted with cells, the actual concentration of compound and enzyme substrate in the cell may be unknown. For example, not all of the liposomes added to an incubation will fuse with the cells and there may be incomplete delivery of the encapsulated compounds. In some embodiments, a tracer, such as, a fluorescent compound, can be included in the liposome. The level of tracer can be used as a means to confirm delivery of the liposomal contents into the cell and to estimate the concentration of the compound and enzyme substrate in the cell interior. In some embodiments, the level of tracer that is retained in a cell can be determined after a wash step to remove undelivered tracer. An example of a suitable tracer is fluorescently labeled insulin, in some embodiments, it may be assumed that the uptake of the compound and enzyme substrate is proportional to the uptake of tracer. Cellular volumes can be measured using conventional techniques, and the internal concentration of a tracer can he estimated and equated to the internal concentration of the compound. To facilitate detection, the tracer can have a label that is distinguishable from that of the enzyme substrate or the substrate products resulting from reaction of substrate with enzyme.

The rate of the reaction catalyzed by the enzyme acting on the enzyme substrate can be determined by monitoring the progress of the signal change over time. The signal can he proportional to the amount of product formed. The rate of reaction can be proportional to the amount of enzyme present, so that the rate of reaction provides a measure of the amount of enzyme present. The initial velocity of the enzyme reaction can be obtained as a function of the substrate concentration and various kinetic parameters obtained. The progress of a reaction can be monitored and analyzed (U.S. Pat. No. 6,108,607 and Duggleby, 1995, Methods Enzymol. 249:60).

The methods described herein can be used for investigations relating to a wide variety of cells. Cell types utilized in the methods, compositions, and kits disclosed include eukaryotic (e.g, animals, plants, yeast., fungi) and bacterial. Viable cells that can be used include fresh cells isolated from a living organism, cells grown or cultured in vitro, or cells reconstituted from frozen or freeze-dried preparations. Cells having a cell wall may be used after appropriate measures are taken to remove the cell wall (Constabel, 1982, in ‘Plant Tissue Culture Methods” pp. 38-48, NRCC No. 19876, Nat. Res. Council of Canada, Saskatoon.). Further examples of cells which can be used are primary or established cell lines and other types of embryonic, neonatal or adult cells, or transformed cells (for example, spontaneously- or virally-transformed). These include, but are not limited to fibroblasts, macrophages, myoblasts, osteoclasts, osteoclasts, heinatopoietic cells, neurons, glial cells, primary B- and T-cells, B- and T-cell lines, chondrocytes, keratinocytes, adipocytes hepatocytes, and other cells described herein and known in the art.

In the methods described herein, a fluorescence signal can be detected using conventional methods and instruments, such as, a fluorometer, fluorescence microscope or confocal microscope for example. hi some embodiments, a multiwavelength fluorescence detector can be utilized. The detector can be used to excite the fluorescence labels at one wavelength and detect emissions as multiple wavelengths, or excite at multiple wavelengths and detect at one emission wavelength. Alternatively, the sample can be excited using “zero-order” excitation in which the full spectrum of light (e.g, from xenon lamp) illuminates the sample. Each label can absorb at its characteristic wavelength of light and then emit maximum fluorescence. The multiple emission signals can be detected independently. Preferably, a suitable detector can be programmed to detect more than one excitation emission wavelength substantially simultaneously, such as, that commercially available under the trade designation HP1100 (G1321A) (Hewlett Packard, Wilmington, Del.). Thus, the fluorescent products can be detected at programmed emission wavelengths at various intervals during a reaction.

In methods herein, cells are allowed to incubate for sufficient time so as to have a sufficient time to inhibit expression of target protein and sufficient turnover of enzyme substrate to produce a light detectable signal. The signal may be observed in a variety of ways. For example, aliquots may be taken and used for fluorescence activated cell sorting (FACS), flow cytotluorometry or static cytofluorometry in a microscope or similar static device. In this manner, a distribution will be obtained for the various levels of fluorescence in the various cells, where the population acts in a heterogeneous manner. The total number of fluorescent cells may be determined where only a fraction of the total cells are infected to provide a particle count. Alternatively, or in combination, total fluorescence may be integrated at different times, so that an overall value may be obtained and the rate of change of the total fluorescence in the cells determined. The background value may be subtracted by employing controls, so that the increase in number of fluorescent cells and fluorescence per cell over time of the cell population may be determined and related to the factor of interest. Alternatively, the cells may be spread on a slide and a fluorescence microscope with an associated fluorometer employed to determine the level of fluorescence of individual cells or groups of cells (e.g, by epifluorescence microscopy). The particular manner in which fluorescence is determined for the cells in the assay is not critical and will vary depending upon available equipment, the qualitative or quantitative nature of the assay, and the like. General descriptions of cell sorting apparatus are provided in U.S. Pat. Nos. 4,172,227; 4,437,935; 4,661,913; 4,667,830; 5,093,234; 5,094,940; 5,144,224; and 6,566,508.

An example of a detection system useful in the present enzyme assay methods is the 8200 Cellular Detection System (Applied Biosystems, an Applera Corporation business). This system is a macro-confocal system based on fluorometric microvolume assay technology (FMAT) that utilizes laser scanning to excite fluorophore contained within cells. The system can differentiate between background fluorescence and that associated with cells and includes multiplexing and automated high-throughput capabilities.

Chemiluminescence can he detected using any of a variety of detectors: Non-limiting example of suitable detectors include luminometers (e.g, Veritas™ Microplate luminometer, Promega; TD-20/20 luminometer, Turner Design, Sunnyvale, Calif.; and BD Moonlight™ 3010 Luminometer, Becton-Dickinson Bioscience), a charge-couple device (CCD) camera, X-ray film, or a scintillation counter.

In some embodiments, light signals can be detected by visual inspection, colorimetry, light microscopy, digital image analyzing, standard microplate reader techniques, video cameras, photographic film. Data can be discriminated and/or analyzed by using pattern recognition software.

The skilled artisan will appreciate that in addition to the cationic liposomes disclosed herein the methods of this disclosure can be performed with other types of liposomes. Essentially any lipid complex that can encapsulate, one or more compounds, including but not limited to compounds capable of of silencing a target protein and an enzyme substrate, and facilitate their delivery into a cell can be used in the present methods, compositions and kits. Essentially any liposome may be used in the methods so long as it is substantially non-toxic to the cell to which it is contacted, at least for the duration of the assay, and is capable of introducing a compound, agent, silencing compound and/or enzyme substrate into the cell under the conditions of the assay. Liposomes may be anionic, cationic or neutral depending upon the choice of hydrophilic group. For instance, when a lipid with a phosphate or a sulfate group is used in the liposome preparation, the resulting Hposotnes will be anionic. When amino-containing lipids are used,. the liposomes will have a positive charge, and will he cationic. When polyethylenoxy or glycol groups are present in the lipid, neutral liposomes are obtained. Additional compounds suitable for forming liposomes may be found in “McCutchen's Detergents and Emulsifiers and McCutchen's functional materials”, Allured Publishing Company, Richwood, N.J., U.S.A.; Lasic, Liposotnes in Gene Delivery, CRC Press, New York pp. 67-112 (1997), Ann. Rev. Biophys. Bioeng. 9:467-508 (1980); European Patent Application 0172007; U.S. Pat. Nos. 4,229,360; 4,241,046; 4,235,871; 4,888,288, 5,455,157; 6,284,538; 6,458,381; and 6,534,018.

Various liposome preparations can include one or more of a variety of lipids, non-limiting examples of which include phosphatidic acid, phosphatidylethanolamine, phosphatidylcholine, phosphatidylinositols, phosphatidylglycerol, sphingomylelin, cardiolipin, phosphatidylserine, cephalin, cerebrosides, dicetylphosphate, steroids, terpenes, acerylpalmitate, glycerol ricinoleate, hexadecyl stearate, isopropyl myristate, amphoteric polymers, triethanolamine lauryl sulfate and cationic lipids, 1-allcyl-2-acyl-phosphoglycerides, and 1-alkyl-1-enyl-2-acyl-phosphoglycerides. In some embodiments, the cationic lipids can include lipids having multiple hydroxy functionalities in the headgroup region, such as, described by Banerjee et al. (J. Med. Chem., 2001, 44:4176-4185). In sonic embodiments, a cationic liposome preparation containing O,O′-ditetradecanoyl-N-(α-trimethylammonioacetyl)diethanolamine chloride, dioleoylphosphatidylethanoiamine and cholesterol (e.g, in a molar ration of 4:3:3 as described by Serikawa, et at Biochim. Biophys, Acta, 2000, 1467:419-430) can be used.

Other amphiphiles useful in forming liposomes include cationic lipids, such as, described in Lasic (1997), pp. 81-86. In some embodiments, one or more of the following lipids may be used in preparing liposomes as described herein: dioctadecyl dimethyl ammonium bromide/chloride (DODA.B/C), dioleoyloxy-3-(trimethylammonio)propane (DOTAP), stearylamine, dodecylamine, hexadecylamine, and dioctadecylamnionium bromide. In some embodiments, the liposomes used herein will contain stearylamine at a mole % that is less than 20%, less than 10%, less than 5%, or less than 1%. In some embodiments, the liposomes are essentially devoid of stearylamine.

A wide variety of suitable lipids are commercially available (such as from Avanti Polar Lipids, Inc. Alabaster, Ala.). Liposome kits are commercially available (e.g., from Boehringer-Mannheim, ProMega, and Life Technologies (Gihco)). Non-limiting examples of suitable lipids include 1,2-dimyristoyl-sn-glycero-3-phosphate (Monosodium Salt) (DMPA·Na) (Avanti catalog no. 830845), 1,2-dimyristoyl-sn-gIycero-3-phosphate (Monosodium Salt) (DOPS·Na) (Avanti catalog no. 830035), and 1,2-dioleoyl-3-trimethylammonium-propane (Chloride Salt) (DTOAP·Cl) (Avanti catalog no. 890890).

Other commercially available liposome kits include LIPOFECTIN, LIPOFECTAMINE™, LIPOFECTACE™, CELLFECTIN™, TRANSFECTAM™, TRX-50™, DC-CHOL™ and DOSPER™ (e.g, as described in Lasic, p. 86).

The liposomes can also include synthetic lipid compounds, such as, D-erythro (C-18) derivatives including sphingosine, ceramide derivatives, and sphinganine; glycosylated (C18) sphingosine and phospholipid derivatives; D-erythro (C17) derivatives; D-erythro (C20) derivatives; and L-threo (C18) derivatives, all of which are commercially available (Avanti Polar Lipids).

Liposomes can include or be wholly formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally includes an ester moiety (ester of a fatty acid), the ester linkage can be replaced with an ether linkage. In some embodiments, lipophilic fluorescent dyes can be embedded non-covalently within the lipid phase of a liposome to assess the integrity of the liposome or to detect the fusion of the liposome with the cell outer membrane. Suitable examples of a lipophilic dye include LAURDANand PATMAN, as described herein. (U.S. Pat. No. 6,569,631). In some embodiments, a membrane impermeable fluorescent dye can be encapsulated along with substrate in a liposome and can act as a tracer to detect fusion and delivery of the liposomal contents into a cell. Examples of such tracer are rhodamine-dextran and fluorescently labeled inulin (U.S. Pat. No. 6,423,547). Lipophilic dyes or tracers can be selected to have spectral characteristics that do not interfere with the detection of the substrates as described herein.

In some embodiments, ‘fusion proteins can be incorporated into the liposome to form a fusigenic liposome as described herein. In some embodiments, liposomes can include cholesterol. Cholesterol intercalates within the phosphatidylcholine bilayer with very little change in area by occupying the regions created by the bulky phosphatidylcholine headgroups. This increases the packing density and structural stability of the bilayer (New, R.R.C., 1990 In New, R.R.C. (ed): Liposomes: a practical approach, Oxford University Press, New York, pp 19-21). The concentration of cholesterol in liposomes can be in the range, for example, of about 5 to about 60 mol %, although higher or lower concentrations can be used.

The composition of the lipid mixture can be selected based on a variety of factors including cost, transition temperature of the lipids, stability during storage, and stability of the liposomes under the reaction conditions. The composition can be selected based upon the compatibility of the liposome with the cell being analyzed.

In some embodiments, lipids for forming liposomes are phospholipid-related materials, such as, lecithin, lysolethicin, phosphatidylinositol, sphingomyelin, cephalin, cardiolipin, phosphatidic acid, cerebrosides, and dicetylphosphate. Additional non-phosphorous containing lipids include, e.g, acetyl palmitate, glycerol ricinoleate, hexadecyl stereate, isopropyl myristate, amphoteric acrylic polymers, alkylaryl sulfate polyethyloxylated fatty acid amides, and the like. In some embodiments, lipids can comprise one or more of: phosphatidylethanolamine, lysophosphatidylethanolamine, phosphatidylserine, stearylamine, dodecylamine, hexadecylamine, triethanolamine-lauryl sulfate.

Another type of liposomal composition includes phospholipids other than naturally derived phosphatidylcholine. Neutral liposome compositions, for example, can be formed from dimyristoyl phosphatidyleholine (DNIPC) or dipaltnitoyl phosphatidylcholine (DPPC). Anionic liposome compositions generally are formed from dimyristoyl phosphatidyiglycerol, while anionic fusogenic liposomes are formed primarily from dioleoyl phosphatidylethanolamine (DOPE). Another type of liposornal composition is formed from phosphatidylcholine (PC) such as, for example, soybean PC, and egg PC. Another type is formed from mixtures of phospholipid and/or phosphatidylcholine and/or cholesterol.

5.4 Kits

In another aspect, kits for delivering one or more compounds or agents to cell are provided. In some embodiments, an agent is a therapeutic agent, a diagnostic agent, a target protein silencing compound, an enzyme substrate or combinations thereof. One or more of the following components may be included in the kit: lipids, phospholipids, cationic Liposomes, eaticmic liposomes containing at least one compound or agent as described herein. In some embodiments, a kit contains a charge neutral compound and/or a charge neutral mixture of compounds and a cationic phospholipid. In some embodiments, a kit contains instructions to generate a cationic liposome capable of delivering a therapeutic agent, diagnostic agent, a target protein silencing compound, an enzyme substrate or combinations to a cell. In some embodiments, kits may have a single container which contains the components described herein or may have distinct container for each component. The components of the kit may be pre-compIexed or each component may be in a separate distinct container.

In some embodiments, a kit can include a lyophilized liposomes preparation, such as, a cationic liposome preparation. In some embodiments, cell viability can be decreased by less than 20%, less than 10%, less than 5% or less than when the liposomes are contacted with cells under conditions described herein.

In some embodiments, a kit can be for detecting an activity or apparent activity of a target protein in a live cell. A kit may have a single container which contains the compounds described herein with or without other components or may have distinct container for each component. The components of the kit may be pre-complexed or each component may be in a separate distinct container. The kit can comprise one or more of the following: liposomes or lipids to form a liposome; a compound capable of silencing a target protein as described herein; an enzyme substrate as described herein, wherein the substrate is capable of producing a light-detectable signal when acted on by an enzyme in a cell; Liposomes comprising a compound capable of silencing a target protein; liposomes comprising compound capable of silencing a target protein and one enzyme substrate as described herein. In some embodiments the kit comprises a lipid capable of forming a liposome comprising a compound capable of silencing. a target protein and an enzyme substrate capable of producing a detectable signal when modified by an enzyme. In some embodiments the liposomes can be cationic. In some embodiments the kit comprises a cationic phospholipid, such as, 1,2-diacylesn-glyeero-3-aikylphosphocholine. In some embodiments, the liposomes of the kit can be included as a lyophilized preparation. In some embodiments, the liposomes are characterized in that cell viability is decreased by less than 20%, less than 10%, less than 5% or less than 1% when the liposomes are contacted with cells under conditions as described herein. The kit can include a modulator (e.g, an inhibitor or an activator) of an enzyme. The kit can further include instructions for carrying out the methods as described herein. The kit can additionally include a cell or cell preparation, a reagent for determining cell viability, and media for suspending cells as described herein. In some embodiments, the media comprises serum. In some embodiment, the kit further comprises serum. In some embodiments, the serum is fetal bovine serum.

All literature and similar materials cited in this application, including but not limited to, patents, patent applications, articles, books, treatises, and the like, regardless of the format of such literature and similar materials, are expressly incorporated by reference in their entirety for any purpose. In the event, that one or more of the incorporated literature and similar materials differs from or contradicts this application, including but not limited to defined terms, term usage, described techniques or the like, this application controls.

The section headings used herein are for organizational p oses only and are not to be construed as limiting the subject matter described in any way.

Aspects of the present teachings may be further understood in light of the following Examples, which should not be construed as limiting the scope of the present teachings in any way. The present teachings encompass various alternatives, modifications, and equivalents.

### 6. EXAMPLES

Aspects of the present teachings may be further understood in light of the following Examples, which should not be construed as limiting the scope of the present teachings in any way.

### Example 1

### Preparation of Cationic Liposomes Encapsulating Labeled Phalloidin

Fluorescently labeled phalloidin was encapsulated in cationic liposomes comprising either a 1:1 ratio of EDOPC:DOPC or a 1:2 ratio of EDOPC:DOPC. Large unilamellar vesicles (LUV) of diameter 100 nm were prepared by the extrusion method essentially as described by Chatterjee et al. (in Methods in Molecular Bioloa: Liposome Methods and Protocols (S. Basu and M. Basu eds.), Humana Press, 2002, vol. 199, chapter 1). Sterile techniques were used throughout this procedure to prevent bacterial contamination of the liposomes. 1,2-Dioleoyl-se-Glycero-3-Phosphocholine (DOPC) (10 mg. Avanti Polar Lipids), and 1,2-Dioleoyl-sn-Glycero-3-Ethylphosphocholine (EDOPC) 10 mg), Avanti Polar Lipids) at a 1:1 ratio were dissolved in chloroform (5 ml) in a 25 ml recovery flask. 1,2-Dioleoyl-en-Glycero-3-Ethylphosphocholine (EDOPC) 5 mg) 1,2-Dioleoyl-sn-Glycero-3-Phosphocholine (DOPC) (10 mg), and at a 1:2 ratio were dissolved in chloroform (5 ml) in as 25 ml recovery flask. The solvent was thoroughly evaporated under high vacuum to leave a thin film, Alexa Fluor 488 phalloidin (600 units, Molecular Probes catalog no. A12379) was added to sterile filtered PBS buffer (2 ml, pH7.2). The Alexa Fluor 488 phalloidin in PBS was added to the lipids and the suspension was subjected to five cycles of freezing (−78° C., dry ice acetone bath) under argon and thawing (40° C.) to hydrate the lipids. The resulting large multilamellar vesicles (LMV) were extruded ten times through two stacked 100 nm polycarbonate membranes (Nuclepore track-etch membrane, Whatman, catalog no. 110605) using a Lipex™ Extruder (Northern Lipids, Inc., British Columbia, Canada, catalog no. T.001). The LUV were purified by Sephadex™ G-25 M gel filtration (PD-10 column, Amersham Biosciences, catalog no. 17-0851-01) eluting with PBS. The liposome size and dispersity was determined by dynamic light scattering using a Nicomp 370 particle size analyzer (Lee Miller, Fine Particle Technology,. Menlo Park, Calif.). The concentration of Alexa Fluor 488 phalloidin within the liposomes was estimated to be 30 units/ml.

### Example 2

### Delivery of Labelled Phalloidin to Live HeLa Cells with Cationic Liposomes Comprising Either a 1:1 Molar Ratio of EDOPC:DOPC or a 1:2 Molar Ratio of EDOPC:DOPC

Approximately 40,000 HeLa cells (ATCC, catalog no. CCL-2) were seeded on poly-L-lysin treated 0.2 mm coverslips in Eagle's minimum essential medium (ATCC, catalog no. 30-2003) containing 10% FBS and 1% pen/strep in a 12 well microtiter plate (Coming, catalog no. 3513). After overnight incubation at 37° C. under 5% CO₂ liposomes comprising a 1:1 molar ratio of EDOPC:DOPC with encapsulated Alexa Fluor 488 phalloidin were prepared in the appropriate cell medium and then added to the cells (1 ml/well) for a final 1:10 dilution of liposomes. Liposomes comprising a 1:2 molar ratio of EDOPC:DOPC with encapsulated phalloidin were prepared in the appropriate cell medium and then added to the cells (1 ml/well) for a final 1:10 dilution of liposomes. After incubation for 2 hr at 37° C. under 5% CO₂ the staining solutions were removed carefully and the cells were washed three times with Dulbecco's PBS (1 ml, ATCC, catalog no. 30-2200). The cells were fixed with 2% paraformaldehyde for 10 min at room temperature. After the fixation the cells were washed twice with 1 ml Dulbecco's PBS. The coverslips were removed from the 12 well plate and mounted on glass slides in AquaPolyMount mounting solution. Cells were analyzed under a fluorescence microscope (Ziess Axiovert 200 M).

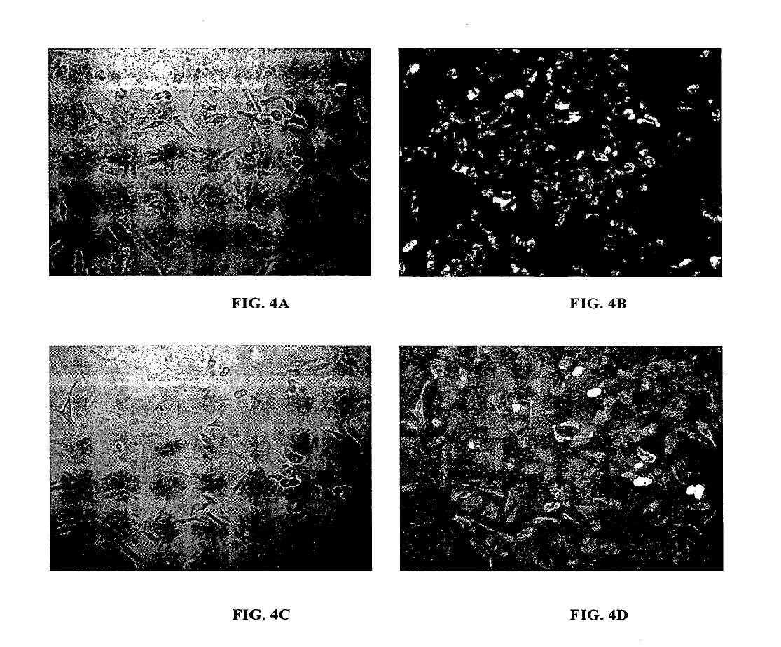

FIGS. 4A and C show HeLa cells contacted with phalloidin encapsulated in cationic liposome comprising either a 1:1 molar ratio of EDOPC:DOPC (FIG. 4A) or 1:2 molar ratio of EDOPC:DOPC (FIG. 4C). Cell viability was determined by observing cell morphology under white light. As can be seen in FIG. 4C the morphology of the HeLa cells contacted with liposomes comprising a 1:2 molar ratio of EDOPC:DOPC appears more normal than cells treated with liposomes comprising the 1:1 molar ratio of EDOPC:DOPC. The HeLa cells in FIG. 4C appear more polygonal and more intact than the HeLa cells in FIG. 4A which appear smaller, rounder and more detached.

FIGS. 4B and 4D show the fluorescent signal produced in HeLa cells contacted with labeled phalloidin encapsulated in a liposome comprising either a 1:1 molar ratio of EDOPC:DOPC (FIG. 4B) or 1:2 molar ratio of EDOPC:DOPC (FIG. 4D). In each of FIGS. 4B and 4D, the cells were excited using 175W Xenon-arc lamp (Sutter Instrument) and a Piston GFP bandpass filter (Chroma Technology Corporation, part no. 41025; exciter: HQ470140; Emitter: HQ515/30). Contacting the cells with liposomes encapsulating phalloidin led to the generation of a detectable fluorescent signal. As can be seen in FIG. 4D, in cells treated with 1:2 molar ratio of EDOPC:DOPC encapsulated phalloidin (FIG. 4D) the cytoskeleton filaments staining appears more uniform than the cells treated with phalloidin encapsulated in 1:1 molar ratio of EDOPC:DOPC (FIG. 4B) , demonstrating the superior ability of 1:2 EDOPC:DOPC liposomes to introduce labeled phalloidin into cells.

### Example 3

### Delivery of Labelled Phalloidin to Live HeLa Cells with Cationic Liposomes Comprising a 1:2 Molar Ratio of EDOPC:DOPC

A pproximately 60,000 HeLa cells (ATCC, catalog no. CCL-2) were seeded on poly-L-lysin treated 0.2 mm coverslips in Eagle's minimum essential medium (ATCC, catalog no. 30-2003) containing 10% FBS and 1% pen/strep in a 6 well microliter plate (Corning, catalog no. 3506). After overnight incubation at 37° C. under 5% CO₂, liposomes comprising a 1:2 molar ratio of EDOPC:DOPC with encapsulated Alexa Fluor 488 phalloidin were prepared in the appropriate cell medium and then added to the cells (2 ml/well) for a final 1:50 dilution of liposomes. After incubation for2 hr at 37° C. under 5% CO₂ the medium was removed carefully and the cells were washed three times with Dulbecco's PBS (2 ml, ATCC, catalog no. 30-2200). After the wash, the cells were stained with 1 μg/ml Hoechst 33258 (Molecular Probes, cat no. H-3569) solution in complete growth medium (2 ml/w) for 10 min at 37° C. under 5% CO₂. The Hoechst solution was removed and the cells were washed three times with 2 ml/w Dulbecco's PBS. The cells were fixed with 2% paraformaldehyde for 10 minutes at room temperature. After the fixation the cells were washed twice with 2 ml/w Dulbecco's PBS. The coverslips were removed from the 12 well plate and mounted on glass slides in AquaPolyMount mounting solution. Cells were analyzed under a fluorescence microscope (Ziess Axiovert 200 M. 63× oil immersion objective, 175W Xenon-arc lamp (Sutter Instrument)). A Piston GFP Bandpass filter (cat. no. 41025 exciter HQ470/40, emitter HQ515/30, Chroma Technology Corporation) was used to detect the phalloidin staining of actin filaments (green) and an 1100v2 UV set filter (Chroma Technology Corporation, excited D360/40×, emitter E420LPv2) was used to detect the Hoechst staining of the nuclei (blue).

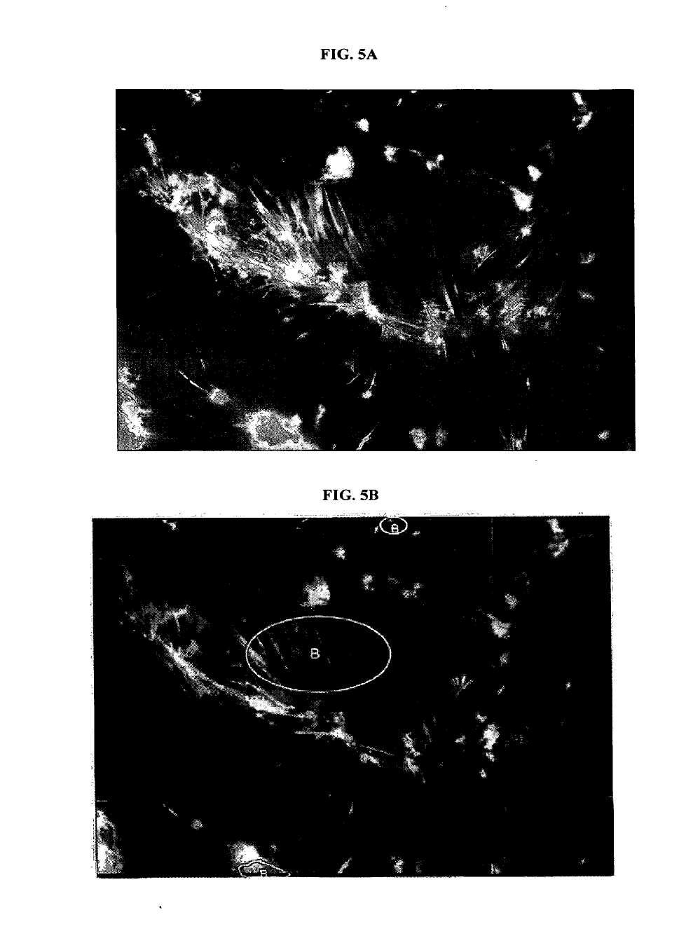

FIG. 5 shows the fluorescent signal produced in HeLa cells contacted with phalloidin encapsulated in a liposome comprising a 1:2 molar ratio of EDOPC:DOPC and Hoechst nuclear staining (blue). Contacting the cells with cationic liposomes encapsulated phalloidin led to the generation of a detectable fluorescent signal as can be seen by phalloidin (green) staining of actin filaments (FIG. 5). The experiment demonstrates the ability of 1:2 EDOPC:DOPC liposomes to introduce phalloidin into live cells.

What is claimed is:

1. A cationic liposome comprising a charge neutral compound andior a charge neutral mixture of compounds and a cationic phospholipid wherein the molar ratio of charge neutral compound and/or a charge neutral mixture of compounds to cationic phospholipid is greater than about 1:1.

2. The liposome of claim 1 in which the charge neutral compound and the charge neutral mixture of compounds are independently selected from the group consisting of phospholipids, lipids, cholesterol, steroids, pegylated phospholipids, tocopherol, nitroxides, and combinations thereof.

3. The liposome of claim 2 in which the lipid is sphingosine, ceramide, cerebroside or derivatives thereof.

4. The liposome of claim 2 in which the phospholipid is a derivative of a (mono/di)radylglycerophospho-monohydroxy alcohol, a (mono/di)radylglycerophospho-polyol, a (mono/di)radylglyceroglycoside, a (monoldi)radyigiycerophosphoglycoside, sphingosine, a phosphoric derivative of a (monoidi)radylglyeerophospho-monohydroxy alchohol, a (monoidi)radylglycerophospho-polyol, a (mono/di)radylglyceroglycoside, a (mono/di)radylglyeerophosphoglycoside or combinations thereof.

5. The liposome of claim 2 in which the phospholipid is an acylphophatidylethanolamine, lysophophatidylethanolamine, acylphophatidyicholine, lysophophatidylcholine, acylphophatidylserine, lysophophatidylserine, acyiphophatidylglycerol, lysophophatidylglycerol, acylphophatidie acid, lysophophatidic acid, acylphophatidylinositol lysophophatidylinositol, acylphophatidylinosito1-4-phosphate, lysophophatidylinositol-4-phosphate, lysophophatidylinositol4,5-diphosphate, aeylphophatidylinosito1-4,5-diphosphate or sphingomyelin

6. The liposome of claim 2 in which the phospholipid is a compound of structural Formula (1): 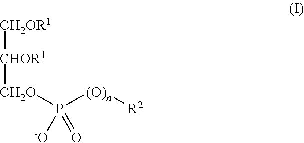
or salts, solvates or hydrates thereof wherein:
each R¹ is independently hydrogen, alkyl or acyl;
n is 0 or 1;
R² is hydrogen, —CH₂CH₂N(CH₃)₃, —CH₂CH₂NH₃, —CH₂CH(CO₂—)NH₃⁺, —CH₂CH(OH)CH₂OH or 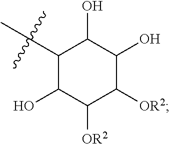
each R² is independently hydrogen or —PO₃H and
with the proviso that at least one R¹ is not hydrogen and is (C₁₀-C₃₀)alkyi or (C₁₀-C₁₀)acyl.

7. The liposome of claim 5 in which n is 1 and R² is hydrogen, —CH₂CH₂N(CH₃)₃, —CH₂CH₂NH₃, —CH₂CH(CO₂—)NH₃⁺, or —CH₂CH(OH)CH₂OH.

8. The liposome of claim 5 in which n is 1 and R¹ is alkyl or acyl.

9. The liposome of claim 5 in which in which n is 1 and one R¹ is hydrogen.

10. The liposorne of claim 5 in which a is 1 and the R¹ group attached to the secondary hydroxyl is hydrogen.

11. The liposome of claim 5 in which each R¹ is identical.

12. The liposome of claim 5 in which each R¹ is independently selected from the group consisting of capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, tricosanoyl, lignoceroyl, rnyristoleoyl, myristelaidoyl, palmitoleoyl, palmitelaidoyl, petroselinoyl, oleoyl, elaidoyl, linoleoy I, linolertoyl, eicosenoyl, arachidonoyl, erucoyl and nervonoyl.

13. The liposome of claim 5 in which the phospholipid is 1,2-clioleoyl-sn-glycero-3-phosphocholine.

14. The liposome of claim 1 in which the cationic phospholipid is a protected (mono/di)radylglycerophospho-monohydroxy alchohol, a protected (mono/di)radylglyceroglycoside, a protected (mono/di)radylglycerophosphoglycoside, sphingosine, a protected pho.sphono derivative of a (monoidi)radylglyeerophospho-monohydroxy alchohol, a protected (mono/di)radylgiyceroglycoside, a protected (mono/di)radylglycerophosphoglycoside or combinations thereof.

15. The liposome of claim 1 in which the cationic phospholipid is a protected acylphophatidylethanolamine, lysophophatidylethanolamine, acylphophatidylcholine, lysophophatidylcholine, acyIphophatidylserine, lysophophatidylserine, acylphophatidylglyceroI, lysophophatidylglycerol, acylphophatidic acid, lysophophatidic acid, acylphophatidylinositol lysophophatidylinositol, acylphophatidylinositol-4-phosphate, lysophophatidylinosinil-4-phosphate, lysophophatidylinositol-4,5-diphosphate, acyIphophatidylinositol-4,5-diphosphate or sphingomyelin.

16. The liposome of claim 1 in which the cationic phospholipid is a compound of structural formula (11): 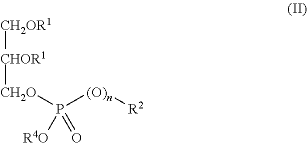
or salts, solvates or hydrates thereof wherein
each R¹ is independently hydrogen, alkyl or acyl;
a is 0 or 1;
R² is —CH₂CH₂N(CH₃)₃, —CH₂CH₂NH₃, and
R⁴ is a protecting group;
with the proviso that at least one R¹ is not hydrogen and is (C₁₀-C₃₀)alkyl or (C₁₀-C₃₀)acyl.

17. The liposome of claim 16 in which the protecting group is —R⁵, —CH₂OC(O)R⁵ or —CH₂CH₂SC(O)R⁵ wherein R⁵ is (C₁-C₆)alkyl.

18. The liposome of claim 17 in which R⁵ is methyl or ethyl.

19. The liposome of claim 15 in which n is 1 and R² is —CH₂CH₂N(CH₃)₃.

20. The liposome of claim 15 in which n is 1 and R¹ is alkyl or acyl.

21. The liposome of claim 15 in which in which n is 1 and one R¹ is hydrogen.

22. The liposome: of claim 15 in which n is 1 and the R¹ group attached to the secondary hydroxyl is hydrogen.

23. The liposome of claim 15 in which R¹ is identical.

24. The liposome of claim 15 in which each le is independently selected from the group consisting of capryl, undecanoyl, lauroyl, tridecartoyl, myristoyl, pentadecanoyl, palmitoyl, phytanoyl, heptaclecanoyl, stearoyl, nonadecanoyl, arachidoyl, heneicosanoyl, tricosanoyl, lignoceroyl, myristoleoyl, myristelaidoyl, palrnitoleoyl, palmitelaidoyl, petroselinoyl, oleoyl, elaidoyl, linoleoyl, linolenoyl, eicosenoyl, arachidonoyl, crucoyl and nervonoyl.

25. The liposome of claim 15 in which n is 1, R¹is alkyl or acyl and R² is —CH₂CH₂N(CH₃)₃.

26. The liposome of claim 15 in which the cationic phospholipid is 1,2-dioleoyl-sn-glycero-3-ethylphosphocholine.

27. The liposome of claim 1 in which the charge neutral compound is 1,2-dioleoyl-sn-glycero-3-phosphocholine and the cationic phospholipid is 1,2-dioleoyl-sn-glycero-3-ethylphosphochoiine.

28.-104. (canceled)
